SEQUENCE LISTING (2 sequences)

SEQ ID NO: 1 - DNA, 37 bp, Artificial Sequence
  Forward primer hCCR5Xbal
agctagtctagatccgttcccctacaagaaactctcc

SEQ ID NO: 2 - DNA, 37 bp, Artificial Sequence
  Reverse primer hCCR5Xbal
agctagtctagagtgcacaactctgactgggtcacca

(19) United States
(12) Patent Application Publication
(10) Pub. No.: US 2006/0019977 A1
(43) Pub. Date: Jan. 26, 2006

(54) Spiroheterocyclic derivative compounds and drugs comprising the compound as the active ingredient

(75) Inventors: Hiromu Habashita, Osaka (JP); Yoshikazu Takaoka, Osaka (JP); Shiro Shibayama, Osaka (JP)

Correspondence Address: SUGHRUE MION, PLLC, 2100 PENNSYLVANIA AVENUE, N.W., SUITE 800, WASHINGTON, DC, 20037, US

(73) Assignee: ONO PHARMACEUTICAL CO., LTD., Osaka (JP)

(21) Appl. No.: 10/531,661

(22) PCT Filed: Oct. 18, 2002
(86) PCT No.: PCT/JP02/10828

§ 371 Date: Apr. 15, 2005

(51) Int. Cl. A61K 31/4747; C07D 471/12

(52) U.S. Cl. 514/278; 546/16

(57) ABSTRACT

Spiroheterocyclic ring derivatives of the formula (I) 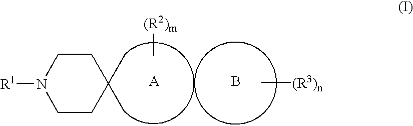
(wherein R¹ is H, alkyl, alkenyl, etc.; R² is keto, thioketo, alkyl, etc.; R³ is alkyl, alkenyl, etc.; AB ring is fused bi-cyclic ring or spiro ring), quaternary ammonium salts thereof, N-oxides thereof, non-toxic salts thereof, or pharmaceutical compositions comprising them, as an active ingredient. The compounds of the formula (I) regulate the effect of chemokine/chemokine receptor, they are used for prevention and treatment of various inflammatory diseases, allergic diseases, immunosuppression, cancer metastasis and acquired immune deficiency syndrome etc.

### TECHNICAL FIELD

The present invention relates to spiroheterocyclic ring derivatives and pharmaceutical compositions comprising thereof, as an active ingredient.

More particularly, it relates to spiroheterocyclic ring derivatives of the formula (I) 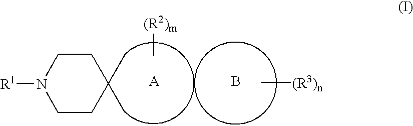
(wherein all the symbols have the same meaning as defined hereinafter), quaternary ammonium salts thereof, N-oxides thereof, non-toxic salts thereof, the methods for preparation thereof and pharmaceutical compositions comprising thereof, as an active ingredient.

### BACKGROUND ART

Chemokine is known as a basic protein having endogeneous leukocyte chemotactic and activating abilities and strong heparin-binding ability. At present, it is considered that chemokine is related to not only the control of infiltration of specific leukocyte at the time of inflammations and immune responses but also the development and homing of lymphocyte under physiological conditions and migration of hemocyte precursor cells and somatic cells.

Differentiation, proliferation and cell death of hemocytes are controlled by various types of cytokine. In the living body, inflammations are found topically and differentiation, maturation and the like of lymphocytes are carried out at certain specified sites. That is, various necessary cells migrate into certain specified sites and accumulate therein to cause a series of inflammations and immune responses. Accordingly, migration of cells is also an indispensable phenomenon in addition to differentiation, proliferation and death of cells.

Migration of hemocytes in the living body starts firstly in the development stage by the shift of hematopoiesis started in the AGM region into permanent hematopoiesis in bone marrow via fetal liver. Furthermore, precursor cells of T cells and thymus dendritic cells migrate from the fetal liver into the bone marrow and then into the thymus gland and cytodifferentiate under thymus environment. The T cell which received clone selection migrates into secondary lymphoid tissues and takes part in an immune response in the periphery. The Langerhans' cell of the skin activated and differentiated by capturing an antigen migrates into the T cell region of a topical lymph node and activates naïve T cell therein as a dendritic cell. The memory T cell performs its homing again into the lymph node via lymphatic and blood vessels. Also, B cell, T cell in the intestinal epithelium, γδ T cell, NKT cell and dendritic cell migrate from bone marrow without passing through the thymus gland and differentiate to take part in an immune response.

Chemokine is deeply related to the migration of these various cells. For example, MIP3β, SLC and its receptor CCR7 play an important role in the migration and homing of naïve T cell, memory T cell and the mature dendritic cell which captured an antigen into a topical lymphoid tissue for the dendritic cells to encounter efficiently with the T cells. The T cell and dendritic cell necessary for controlling antigen-specific immune responses are hardly observed in the secondary lymph node of a PLT mouse having deficiency in the expression of SLC [J. Exp. Med., 189(3), 451 (1999)].

MDC, TARC and its receptor CCR4 play an important role in the migration of Th2 cell into topical sites in immune and inflammatory responses in which the Th2 cell is related. In a rat fluminant hepatitis model (P. acnes+LPS), an anti-TARC antibody suppressed increase of the amount of ALT in blood and increase of the expressing amounts of TNFα and FasL in the liver and also improved lethality of the rats [J. Clin. Invest., 102, 1933 (1998)]. Also, an anti-MDC antibody decreased the number of eosinophils accumulated in the lung interstitium and suppressed airway hypersensitivity in a mouse OVA-induced airway hypersensitivity model [J. Immunology, 163, 403 (1999)].

MCP-1 and its receptor CCR2 are related to the infiltration of macrophage into inflammation sites. An anti-MCP-1 antibody showed an effect to suppress infiltration of monocyte and macrophage into glomerulus in a rat anti-Thy1.1 antibody glomerular nephritis model [Kidney Int., 51, 770 (1997)].

Thus, chemokine receptors are greatly related to the control of inflammation and immune responses through a mechanism in which they are expressed at certain specified periods in variously specific cells and the effector cells are accumulated in a region where chemokine is produced.

Acquired immunodeficiency syndrome (called AIDS) which is induced by human immunodeficiency virus (hereinafter referred to as “HIV”) is one of the diseases of which their therapeutic methods are most earnestly desired in recent years. Once infection with HIV is completed in a CD4-positive cell which is a principal target cell, HIV repeats its proliferation in the body of the patient and, sooner or later, completely destroys T cell which takes charge of the immunological function. During this process, the immunological function is gradually reduced to cause fever, diarrhea, lymph node enlargement and the like various immunodeficiency conditions which are apt to cause complications with pneumocystis carinii pneumonia and the like various opportunistic infections. Such conditions are the onset of AIDS, and it is well known that they induce and worsen Kaposi sarcoma and the like malignant tumors.

As the recent preventive and therapeutic methods for AIDS, attempts have been made to, e.g., (1) inhibit growth of HIV by the administration of a reverse transcriptase inhibitor or a protease inhibitor and (2) prevent or alleviate opportunistic infections by the administration of a drug having immunopotentiation activity.

Helper T cells which take charge of the central of immune system are mainly infected with HIV. It is known since 1985 that HIV uses the membrane protein CD4 expressing on the membrane of T cells in the infection [Cell, 52, 631 (1985)]. The CD4 molecule is composed of 433 amino acid residues, and its expression can be found in macrophages, some B cells, vascular endothelial cells, Langerhans' cells in skin tissues, dendritic cells in lymphoid tissues, glia cells of the central nervous system and the like, in addition to the mature helper T cells. However, since it has been revealed that the infection with HIV is not completed by the CD4 molecule alone, a possibility has been suggested on the presence of factors other than the CD4 molecule, which are related to the infection of cells with HIV.

In 1996, a cell membrane protein called Fusin was identified as a factor other than the CD4 molecule, which is related to the HIV infection [Science, 272, 872 (1996)]. It was confirmed that this Fusin molecule is a receptor (namely, CXCR4) of stromal derived factor-1 (hereinafter referred to as “SDF-1”). In addition, it was confirmed also in vitro that the SDF-1 specifically inhibits infection of T cell tropic (X4) HIV [Nature, 382, 829 (1996), Nature, 382, 833 (1996)]. That is, it is considered that the HIV infection was inhibited by the binding of SDF-1 to CXCR4 preceding HIV, thereby depriving HIV of a foothold for infecting cells.

Also at that time, it was discovered that another chemokine receptor CCR5, which is a receptor of RANTES, MIP-1α and MIP-1β, is also used at the time of the infection with a macrophage tropic (R5) HIV [Science, 272, 1955 (1996)].

Accordingly, substances which can compete With CXCR4 and CCR5 for HIV, or which can bind to HIV virus thus causing the virus unable to bind to CXCR4 and CCR5, could become HIV infection inhibitors. Also, there is a case in which a low molecular compound initially discovered as an HIV infection inhibitor was actually a CXCR4 antagonist [Nature Medicine, 4, 72 (1998)].

Based on the above, it is considered that the chemokine/chemokine receptors are deeply related to the inflammation, immune disease or HIV infection. For example, it is considered that they are related to the inhibition of various inflammatory diseases, asthma, atopic dermatitis, nettle rash, allergic diseases (allergic bronchopulmonary aspergillosis, allergic eosinophilic gastroenteritis and the like), glomerular nephritis, nephropathy, hepatitis, arthritis, chronic rheumatoid arthritis, psoriasis, rhinitis, conjunctivitis and ischemia-reperfusion injury, in the treatment of multiple sclerosis, ulcerative colitis, acute respiratory distress syndrome, shock accompanied by bacterial infection, diabetes mellitus and autoimmune diseases, and in transplanted organ rejection reactions, immunosuppression, metastasis prevention and acquired immunodeficiency syndrome.

On the other hand, in specification of WO97/11940, it is described that compounds of the formula (Z) 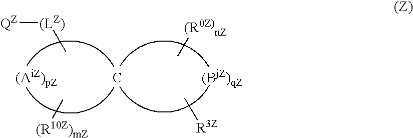
(wherein the atoms A^{iZ} and B^{iZ} are independently selected from carbon, nitrogen, oxygen or sulfur, provided that at least one atom of A^{iZ} is carbon, and at least one atom B^{iZ} is carbon;
each of the rings of the spirobicycle formed by A^{iZ} and B^{iZ} may be partly unsaturated,
pZ and qZ are independently numbers from 2 to 6,
mz is a number from 0 to pZ,
R^{10Z} is the same or different and is a non-interfering substituent independently selected from hydrogen, alkyl, halosubstituted alkyl, alkenyl, alkynyl, cycloalkyl, ═O, ═S etc.,
nZ is a number from 0 to qZ,
R^{0Z} is the same or different and is a non-interfering substituent independently selected from hydrogen, alkyl, halosubstituted alkyl, alkenyl, alkynyl, cycloalkyl, ═0, ═S etc.,
the linking group -(L^{Z})- is a bond or a divalent substituted or unsubstituted chain of from 1 to 10 atoms selected from the group consisting of carbon, nitrogen, sulfur, and oxygen,
Q^{Z} is a basic group containing one or more basic radicals, and
R^{3Z} is an acidic group containing one or more acid radicals) are useful in inhibition platelet aggregation.

In specification of WO98/25605, it is described that compounds of the formula (Y) 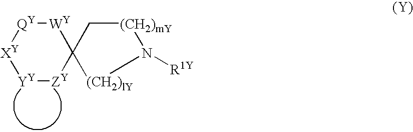
(wherein mY or IY are each independently 0, 1, 2, 3, 4 or 5,
R^{1Y} is hydrogen, C1-8 alkyl, C2-8 alkenyl, C2-8 alkynyl etc.,
W^{Y} is a bond, C1-3 alkyl or C1-3 alkyl substituted with oxo etc.,
Q^{Y} is —NR²—, —O—, —S—, —S(O)— or —SO₂—,
X^{Y} is a bond, C1-3 alkyl or C1-3 alkyl substituted with oxo etc.,
Y^{Y}-Z^{Y} ring is phenyl, naphthyl or hetero aryl, with the proviso that the definition of each symbol is a excerpt partially) are useful as modulators of the chemokine receptors.

### DISCLOSURE OF THE INVENTION

The present inventors have investigated to find compounds regulating chemokine/chemokine receptors, so that the present inventors have found that the purpose has been achieved by spiroheterocyclic ring derivatives of the formula (I).

The present invention relates to
i) spiroheterocyclic ring derivatives of the formula (I) 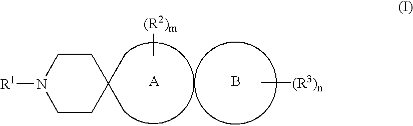
   wherein R¹ is:
   (1) hydrogen,
   (2) C1-18 alkyl,
   (3) C2-18 alkenyl,
   (4) C2-18 alkynyl,
   (5) —COR⁶,
   (6) —CONR⁷R⁸,
   (7) —COOR⁹,
   (8) —SO₂R¹⁰,
   (9) —COCOOR¹¹,
   (10) —CONR¹²COR¹³,
   (11) Cyc 1, or
   (12) C1-18 alkyl, C2-18 alkenyl or C2-18 alkynyl substituted by 1-5 substituent(s) selected from (a) halogen, (b) —CONR⁷R⁸, (c) —COOR⁹, (d) —OR¹⁴, (e) —SR¹⁵, (f) —NR¹⁶R¹⁷, (g) —NR¹⁸COR¹⁹, (h) —SO₂NR²⁰R²¹, (i) —OCOR²², (j) —NR²³SO₂R²⁴, (k) —NR²⁵COOR²⁶ (l) —NR²⁷CONR²⁸R²⁹, (m) Cyc 1, (n) keto or (o) —N(SO₂R²⁴)₂,
   wherein R⁶-R⁹, R¹¹-R²¹, R²³, R²⁵ and R²⁷-R²⁹ are each independently:
   (1) hydrogen,
   (2) C1-8 alkyl,
   (3) C2-8 alkenyl,
   (4) C2-8 alkynyl,
   (5) Cyc 1, or
   (6) C1-8 alkyl, C2-8 alkenyl or C2-8 alkynyl substituted by 1-5 substituent(s) selected from (a) Cyc 1, (b) halogen, (c) —OR³⁰, (d) —SR³′, (e) —NR³²R³³, (f) —COOR³⁴, (g) —CONR³⁵R³⁶, (h) —NR³⁷COR³⁸, (i) —NR³⁹SO₂R⁴⁰ or (j) —N(SO₂R⁴⁰)₂, or
   R⁷ and R⁸, R²⁰ and R²¹, R²⁸ and R²⁹, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁵—(C2-6 alkylene)-,
   R¹⁹⁵ is hydrogen, C1-8 alkyl, phenyl or C1-8 alkyl substituted by phenyl, R¹⁰, R²², R²⁴ and R²⁶ are each independently:
   (1) C1-8 alkyl,
   (2) C2-8 alkenyl,
   (3) C2-8 alkynyl,
   (4) Cyc 1, or
   (5) C1-8 alkyl, C2-8 alkenyl or C2-8 alkynyl substituted by 1-5 substituent(s) selected from (a) Cyc 1, (b) halogen, (c) —OR³⁰, (d) —SR³′, (e) —NR³²R³³, (f) —COOR³⁴, (g) —CONR³⁵R³⁶ (h) —NR³⁷COR³⁸, (i) —NR³⁹SO₂R⁴⁰ or (j) —N(SO₂R⁴⁰)₂,
   R³⁰-R³⁷ and R³⁹ are each independently, hydrogen, C1-8 alkyl, Cyc 1 or C1-8 alkyl substituted by Cyc 1, or
   R³⁵ and R³⁶, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁶—(C2-6 alkylene)-,
   R¹⁹⁶ is hydrogen, C1-8 alkyl, phenyl or C1-8 alkyl substituted by phenyl,
   R³⁸ and R⁴⁰ are each independently C1-8 alkyl, Cyc 1 or C1-8 alkyl substituted by Cyc 1,
   Cyc 1 is a C3-15 mono, bi- or tri-(fused or spiro)carbocyclic ring or a 3-15 membered mono-, bi- or tri-(fused or spiro)cyclic hetero ring containing 1-4 nitrogen atom(s), 1-3 oxygen atom(s) and/or 1-3 sulfur atom(s),
   Cyc 1 may be substituted by 1-5 of R⁵¹,
   R⁵¹ is:
   (1) C1-8 alkyl,
   (2) C2-8 alkenyl,
   (3) C2-8 alkynyl,
   (4) halogen,
   (5) nitro,
   (6) trifluoromethyl,
   (7) trifluoromethoxy,
   (8) nitrile,
   (9) keto,
   (10) Cyc 2
   (11) —OR⁵²,
   (12) —SR⁵³,
   (13) —NR⁵⁴R⁵⁵,
   (14)—COOR⁵⁶,
   (15) —CONR⁵⁷R⁵⁸,
   (16) NR⁵⁹COR⁶⁰
   (17) —SO₂NR⁶, R⁶²,
   (18)—OCOR⁶³,
   (19) —NR⁶⁴SO₂R⁶⁵,
   (20) —NR⁶⁶COOR⁶⁷,
   (21) —NR⁶⁸CONR⁶⁹R⁷⁰,
   (22) —B(OR⁷¹)₂,
   (23) —SO₂R⁷²,
   (24)—N(SO₂R⁷²)₂, or
   (25) C1-8 alkyl, C2-8 alkenyl or C2-8 alkynyl substituted by 1-5 substituent(s) selected from (a) halogen, (b) Cyc 2, (c) —OR⁵², (d) —SR⁵³, (e) —NR⁵⁴R⁵⁵, (f) —COOR⁵⁶, (g) —CONR⁵⁷R⁵⁸, (h) —NR⁵⁹COR⁶⁰, (i) —SO₂NR⁶¹R⁶², (j) —OCOR⁶³, (k) —NR⁶⁴SO₂R⁶⁵, (l) —NR⁶⁶COOR⁶⁷, (m) —NR⁶⁸CONR⁶⁹R⁷⁰, (n) —B(OR⁷¹)₂, (o) —SO₂R⁷², (p) —N(SO₂R⁷²)₂ or (q) keto,
   R⁵²-R⁶², R⁶⁴, R⁶⁶ and R⁶⁸-R⁷¹ are each independently 1) hydrogen, 2) C1-8 alkyl, 3) C2-8 alkenyl, 4) C2-8 alkynyl, 5) Cyc 2 or 6) C1-8 alkyl, C2-8 alkenyl or C2-8 alkynyl substituted by Cyc 2, —OR⁷³, —COOR⁷⁴ or —NR⁷⁵R⁷⁶, or
   R⁵⁷ and R⁵⁸, R⁶¹ and R⁶², R⁶⁹ and R⁷⁰, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁷—(C2-6 alkylene)-,
   R¹⁹⁷ is hydrogen, C1-8 alkyl, phenyl or C1-8 alkyl substituted by phenyl,
   R⁶³, R⁶⁵, R⁶⁷ and R⁷² are each independently 1) C1-8 alkyl, 2) C2-8 alkenyl, 3) C2-8 alkynyl, 4) Cyc 2 or 5) C1-8 alkyl, C2-8 alkenyl or C2-8 alkynyl substituted by Cyc 2, —OR⁷³, —COOR⁷⁴ or —NR⁷⁵R⁷⁶,
   R⁷³-R⁷⁶ are independently hydrogen, C1-8 alkyl, Cyc 2 or C1-8 alkyl substituted by Cyc 2,
   Cyc 2 has the same meaning as Cyc 1,
   Cyc 2 may be substituted by 1-5 of R⁷⁷,
   R⁷⁷ is:
   1) C1-8 alkyl,
   2) halogen,
   3) nitro,
   4) trifluoromethyl,
   5) trifluoromethoxy,
   6) nitrile,
   7) —OR⁷⁸,
   8) —NR⁷⁹R⁸⁰,
   9) —COOR⁸¹,
   10)—SR⁸²,
   11) —CONR⁸³R⁸⁴,
   12) C2-8 alkenyl,
   13) C2-8 alkynyl,
   14) keto,
   15) Cyc 6,
   16)—NR⁶¹COR¹⁶²,
   17)—SO₂NR¹⁶³R¹⁶⁴,
   18)—OCOR¹⁶⁵,
   19) —NR¹⁶⁶SO₂R¹⁶⁷,
   20)—NR¹⁶⁸COOR¹⁶⁹,
   21) —NR¹⁷⁰CONR¹⁷¹R¹⁷²,
   22) —SO₂R¹⁷³,
   23) —N(SO₂R¹⁶⁷)₂, or
   24) C1-8 alkyl, C2-8 alkenyl or C2-8 alkynyl substituted by 1-5 substituent(s) selected from (a) halogen, (b) —OR⁷⁸, (c) —NR⁷⁹R⁸⁰, (d) —COOR⁸¹, (e) —SR⁸², (f) —CONR⁸³R⁸⁴, (g) keto, (h) Cyc 6, (i) —NR¹⁶¹COR¹⁶², (j) —SO₂NR⁶³R¹⁶⁴, (k) —OCOR¹⁶⁵, (l) —NR¹⁶⁶SO₂R¹⁶⁷, (m) —NR¹⁶⁸COOR¹⁶⁹, (n) —NR¹⁷⁰CONR¹⁷¹R¹⁷², (o) —SO₂R¹⁷³ or (p) —N(SO₂R¹⁶⁷)₂,
   R⁷⁸—R⁸⁴, R¹⁶¹-R¹⁶⁴, R¹⁶⁶, R¹⁶⁸ and R¹⁷⁰-R¹⁷² are each independently (a) hydrogen, (b) C1-8 alkyl, (c) C2-8 alkenyl, (d) C2-8 alkynyl, (e) Cyc 6 or (f) C1-8 alkyl, C2-8 alkenyl or C2-8 alkynyl substituted by Cyc 6, —OR¹⁷⁴, —COOR¹⁷⁵, —NR¹⁷⁶R¹⁷⁷ or —CONR¹⁷⁸R¹⁷⁹, or
   R⁸³ and R⁸⁴, R¹⁶³ and R¹⁶⁴, R¹⁷¹ and R¹⁷², taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁸—(C2-6 alkylene)-,
   R¹⁹⁸ is hydrogen, C1-8 alkyl, phenyl or C1-8 alkyl substituted by phenyl,
   R¹⁶⁵, R¹⁶⁷, R¹⁶⁹ and R¹⁷³ are each independently (a) C1-8 alkyl, (b) C2-8 alkenyl, (c) C2-8 alkynyl, (d) Cyc 6 or (e) C1-8 alkyl, C2-8 alkenyl or C2-8 alkynyl substituted by Cyc 6, —OR¹⁷⁴, —COOR¹⁷⁵, —NR⁷⁶R¹⁷⁷ or —CONR¹⁷⁸R¹⁷⁹,
   R¹⁷⁴-R¹⁷⁷ are each independently (1) hydrogen, (2) C1-8 alkyl, (3) Cyc 6 or (4) C1-8 alkyl substituted by Cyc 6, or
   R¹⁷⁸ and R¹⁷⁹, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁹—(C2-6 alkylene)-,
   R¹⁹⁹ is hydrogen, C1-8 alkyl, phenyl or C1-8 alkyl substituted by phenyl,
   Cyc 6 is a C3-8 mono-carbocyclic ring or a 3-8 membered mono-cyclic hetero ring containing 1-4 nitrogen atom(s), 1-2 oxygen atom(s) and/or 1-2 sulfur atom(s),
   Cyc 6 may be substituted by 1-5 of R¹⁸⁰,
   R¹⁸⁰ is:
   (1) C1-8 alkyl,
   (2) halogen,
   (3) nitro,
   (4) trifluoromethyl,
   (5) trifluoromethoxy,
   (6) nitrile,
   (7) —OR¹⁸¹,
   (8) —NR¹⁸²R¹⁸³,
   (9) —COOR¹⁸⁴,
   (10) —SR¹⁸⁵, or
   (11) —CONR¹⁸⁶R¹⁸⁷,
   R¹⁸¹-R¹⁸⁷ are each independently (1) hydrogen, (2) C1-8 alkyl, (3) phenyl or (4) C1-8 alkyl substituted by phenyl, or
   R¹⁸² and R¹⁸³, R¹⁸⁶ and R¹⁸⁷, taken together, are (1) C2-6 alkylene, (2) —(C2-6 alkylene)-O—(C2-6 alkylene)-, (3) —(C2-6 alkylene)-S—(C2-6 alkylene)- or (4) —(C2-6 alkylene)-NR²⁰⁰—(C2-6 alkylene)-,
   R²⁰⁰ is hydrogen, C1-8 alkyl, phenyl or C1-8 alkyl substituted by phenyl, 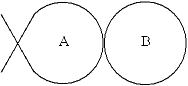
      is (i) a fused bi-cyclic ring which A ring and B ring bound by two atoms or (ii) a spiro ring which A ring and B ring bound by spiro,
   A ring is (i) a C5 or 6 partially or fully saturated carbocyclic ring or (ii) a 5 or 6 membered partially or fully saturated hetero ring containing 1-3 hetero atom(s) selected from a nitrogen atom(s), an oxygen atom(s) and/or a sulfur atom(s),
   B ring is (i) a C4-7 partially or fully saturated carbocyclic ring or (ii) a 4-7 membered partially or fully saturated hetero ring containing 1-3 hetero atom(s) selected from a nitrogen atom(s), an oxygen atom(s) and/or a sulfur atom(s),
   R² is:
   (1) keto,
   (2) thioketo,
   (3) C1-8 alkyl,
   (4) C2-8 alkenyl,
   (5) C2-8 alkynyl,
   (6) —OR⁹⁰,
   (7) Cyc 3, or
   (8) C1-8 alkyl, C2-8 alkenyl or C2-8 alkynyl substituted by 1-5 substituent(s) selected from (a) halogen, (b) —OR⁹⁰, (c) —SR⁹¹, (d) —NR⁹²R⁹³, (e) —COOR⁹⁴, (f) —CONR⁹⁵R⁹⁶, (g) —NR⁹⁷COR⁹⁸, (h) —SO₂NR⁹⁹R¹⁰⁰, (i) —OCOR¹⁰¹, (j) —NR¹⁰²SO₂R¹⁰³, (k) —NR¹⁰⁴COOR¹⁰⁵, (l) —NR¹⁰⁶CONR¹⁰⁷R¹⁰⁸, (m) Cyc 3, (n) keto or (o) —N(SO₂R¹⁰³)₂,
   R⁹⁰-R¹⁰⁰, R¹⁰², R¹⁰⁴ and R¹⁰⁶-R¹⁰⁸ are each independently (1) hydrogen, (2) C1-8 alkyl, (3) C2-8 alkenyl, (4) C2-8 alkynyl, (5) Cyc 3 or (6) C1-8 alkyl, C2-8 alkenyl or C2-8 alkynyl substituted by Cyc 3, or
   R⁹⁵ and R⁹⁶, R⁹⁹ and R¹⁰⁰, R¹⁰⁷ and R¹⁰⁸, taken together, are (1) C2-6 alkylene, (2) —(C2-6 alkylene)-O—(C2-6 alkylene)-, (3) —(C2-6 alkylene)-S—(C2-6 alkylene)- or (4) —(C2-6 alkylene)-NR²⁰²—(C2-6 alkylene)-,
   R²⁰² is hydrogen, C1-8 alkyl, phenyl or C1-8 alkyl substituted by phenyl,
   R¹⁰¹, R¹⁰³ and R¹⁰⁵ are each independently (1) C1-8 alkyl, (2) C2-8 alkenyl, (3) C2-8 alkynyl or (4) Cyc 3, or (5) C1-8 alkyl, C2-8 alkenyl or C2-8 alkynyl substituted by Cyc 3,
   Cyc 3 has the same meaning as Cyc 1,
   Cyc 3 may be substituted by 1-5 of R¹⁰⁹,
   R¹⁰⁹ has the same meaning as R⁵¹,
   R³ is:
   (1) C1-8 alkyl,
   (2) C2-8 alkenyl,
   (3) C2-8 alkynyl,
   (4) —COOR¹²⁰,
   (5) —CONR¹²¹R¹²²,
   (6) Cyc 4, or
   (7) —OR¹²³,
   (8) —COR¹³¹,
   (9) —SO₂R¹³³, or
   (10) C1-8 alkyl, C2-8 alkenyl or C2-8 alkynyl substituted by an substituent(s) selected from (a) halogen, (b) nitrile, (c) Cyc 4, (d) —COOR¹²⁰, (e) —CONR¹²¹R¹²², (f) —OR¹²³, (g) —SR¹²⁴, (h) —NR¹²⁵R¹²⁶, (i) —NR¹²⁷COR¹²⁸, (j) —SO₂NR¹²⁹R¹³¹, (k) —OCOR¹³¹, (l) —NR¹³²SO₂R¹³³, (m) —NR¹³⁴COOR¹³⁵, (n) —NR¹³⁶CONR¹³⁷R¹³⁸ or (o) keto,
   R¹²⁰-R¹³⁰, R¹³², R¹³⁴ and R¹³⁶-R¹³⁸ are each independently (1) hydrogen, (2) C1-8 alkyl, (3) C2-8 alkenyl, (4) C2-8 alkynyl, (5) Cyc 4 or (6) C1-8 alkyl, C2-8 alkenyl or C2-8 alkynyl substituted by Cyc 4, halogen, —OR⁴⁸, —SR¹⁴⁹, —COOR¹⁵⁰ or —NHCOR¹⁴¹, or
   R¹²¹ and R¹²², R¹²⁹ and R¹³⁰, R¹³⁷ and R¹³⁸, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR²⁰²—(C2-6 alkylene)-,
   R²⁰² is hydrogen, C1-8 alkyl, phenyl or C1-8 alkyl substituted by phenyl,
   R¹³¹, R¹³³ and R¹³⁵ are each independently (1) C1-8 alkyl, (2) C2-8 alkenyl, (3) C2-8 alkynyl, (4) Cyc 4 or (5) C1-8 alkyl, C2-8 alkenyl or C2-8 alkynyl substituted by Cyc 4, halogen, —SR¹⁴⁸, —COOR¹⁵⁰ or —NHCOR¹⁴,
   R¹⁴¹ is (1) C1-8 alkyl, (2) C2-8 alkenyl, (3) C2-8 alkynyl, (4) Cyc 4 or (5) C1-8 alkyl, C2-8 alkenyl or C2-8 alkynyl substituted by Cyc 4,
   R¹⁴⁸-R¹⁵⁰ are each independently (1) hydrogen, (2) C1-8 alkyl, (3) C2-8 alkenyl, (4) C2-8 alkynyl, (5) Cyc 4 or (6) C1-8 alkyl, C2-8 alkenyl or C2-8 alkynyl substituted by Cyc 4,
   Cyc 4 has the same meaning as Cyc 1,
   Cyc 4 may be substituted by 1-5 of R¹⁴⁴,
   R¹⁴⁴ has the same meaning as R⁵¹,
   m is 0-5,
   n is 0-5,
   when m is 2-5, then R² of m are the same or different, when n is 2-5, then R³ of n are the same or different, a quaternary ammonium salt thereof, an N-oxides thereof or a non-toxic salt thereof,
ii) processes for the preparation thereof, and
iii) pharmaceutical agents containing such a derivative as an active ingredient.

In the present invention, C1-8 alkyl means methyl, ethyl, propyl, butyl, pentyl, hexyl, heptyl, octyl or isomeric groups thereof.

C1-18 alkyl means methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl or isomeric groups thereof.

C2-8 alkenyl means C2-8 alkylene optionally having 1-4 double bond(s), for example, vinyl, propenyl, butenyl, pentenyl, hexenyl, heptenyl, octenyl, butadienyl, pentadienyl, hexadienyl, heptadienyl, octadienyl, hexatrienyl, heptatrienyl, octatrienyl or isomeric groups thereof.

C2-18 alkenyl means C2-18 alkylene optionally having 1-9 double bond(s) (preferably 1-4 double bond(s)), for example, vinyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, butadienyl, pentadienyl, hexadienyl, heptadienyl, octadienyl, nonadienyl, decadienyl, undecadienyl, dodecadienyl, tridecadienyl, tetradecadienyl, pentadecadienyl, hexadecadienyl, heptadecadienyl, octadecadienyl, hexatrienyl, heptatrienyl, octatrienyl, nonatrienyl, decatrienyl, undecatrienyl, dodecatrienyl, tridecatrienyl, tetradecatrienyl, pentadecatrienyl, hexadecatrienyl, heptadecatrienyl, octadecatrienyl or isomeric groups thereof.

C2-8 alkynyl means C2-8 alkylene optionally having 1-4 triple bond(s) (preferably 1-4 triple bond(s)), for example, ethynyl, propynyl, butynyl, pentynyl, hexynyl, heptynyl, octynyl, butadiynyl, pentadiynyl, hexadiynyl, heptadiynyl, octadiynyl, hexatriynyl, heptatriynyl, octatriynyl, or isomeric groups thereof.

C2-18 alkynyl means C2-18 alkylene optionally having 1-9 triple bond(s) (preferably 1-4 triple bond(s)), for example, ethynyl, propynyl, butynyl, pentynyl, hexynyl, heptynyl, octynyl, nonynyl, decynyl, undecynyl, dodecynyl, tridecynyl, tetradecynyl, pentadecynyl, hexadecynyl, heptadecynyl, octadecynyl, butadiynyl, pentadiynyl, hexadiynyl, heptadiynyl, octadiynyl, nonadiynyl, decadiynyl, undecadiynyl, dodecadiynyl, tridecadiynyl, tetradecadiynyl, pentadecadiynyl, hexadecadiynyl, heptadecadiynyl, octadecadiynyl, hexatriynyl, heptatriynyl, octatriynyl, nonatriynyl, decatriynyl, undecatriynyl, dodecatriynyl, tridecatriynyl, tetradecatriynyl, pentadecatriynyl, hexadecatriynyl, heptadecatriynyl, octadecatriynyl or isomeric groups thereof.

Halogen is chlorine, bromine, fluorine or iodine.

C2-6 alkylene means methylene, ethylene, trimethylene, tetramethylene, pentamethylene, hexamethylene or isomeric groups thereof.

C3-15 mono-, bi- or tri-(fused or spiro)carbocyclic ring means for example, cyclopropane, cyclobutane, cyclopentane, cyclohexane, cycloheptane, cyclooctane, cyclopentene, cyclohexene, cycloheptene, cyclooctene, cyclopentadiene, cyclohexadiene, cycloheptadiene, cyclooctadiene, benzene, indene, naphthalene, indan, tetrahydronaphthalene, bicyclo[3.3.0]octane, bicyclo[4.3.0]nonane, bicyclo[4.4.0]decane, spiro[4.4]nonane, spiro[4.5]decane, spiro[5.5]undecane, bicyclo[3.1.1]heptane, bicyclo[3.3.1]hept-2-ene, fluorene or anthracene ring etc.

The 3-15 membered mono-, bi- or tri-(fused or spiro)cyclic hetero ring containing 1-4 nitrogen atom(s), 1-3 oxygen atom(s) and/or 1-3 sulfur atom(s) means 3-15 membered mono-, bi- or tri-(fused or spiro)cyclic hetero aryl containing 1-4 nitrogen atom(s), 1-3 oxygen atom(s) and/or 1-3 sulfur atom(s), and partially or fully saturated one.

The 3-15 membered mono-, bi- or tri-(fused or spiro)cyclic hetero aryl containing 1-4 nitrogen atom(s), 1-3 oxygen atom(s) and/or 1-3 sulfur atom(s) is pyrrole, imidazole, triazole, tetrazole, pyrazole, pyridine, pyrazine, pyrimidine, pyridazine, azepine, diazepine, furan, pyran, oxepine, thiophene, thiain (thiopyran), thiepine, oxazole, isoxazole, thiazole, isothiazole, furazan, oxadiazole, oxazine, oxadiazine, oxazepine, oxadiazepine, thiadiazole, thiazine, thiadiazine, thiazepine, thiadiazepine, indole, isoindole, benzofuran, isobenzofuran, benzothiophene, isobenzothiophene, indazole, quinoline, isoquinoline, phthalazine, naphthyridine, quinoxaline, quinazoline, cinnoline, benzoxazole, benzothiazole, benzimidazole, benzoxepine, benzoxazepine, benzoxadiazepine, benzothiepine, benzothiazepine, benzothiadiazepine, benzazepine, benzodiazepine, benzofurazan, benzothiadiazole, benzotriazole, carbazole, acridine, dibenzofuran or dibenzothiophene ring etc.

In the above 3-15 membered mono-, bi- or tri-(fused or spiro)cyclic hetero ring containing 1-4 nitrogen atom(s), 1-3 oxygen atom(s) and/or 1-3 sulfur atom(s), partially or fully saturated one is pyrroline, pyrrolidine, imidazoline, imidazolidine, pyrazoline, pyrazolidine, triazoline, triazolidine, tetrazoline, tetrazolidine, dihydropyridine, tetrahydropyridine, piperidine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydroazepine, tetrahydroazepine, perhydroazepine, dihydrodiazepine, tetrahydrodiazepine, perhydrodiazepine, dihydrofuran, tetrahydrofuran, dihydropyran, tetrahydropyran, dihydrothiophene, tetrahydrothiophene, dihydrothiain (dihydrothiopyran), tetrahydrothiain (tetrahydrothiopyran), dihydrooxazole, tetrahydrooxazole, dihydroisoxazole, tetrahydroisoxazole, dihydrothiazole, tetrahydrothiazole, dihydroisothiazole, tetrahydroisothiazole, dihydrooxadiazole, tetrahydrooxadiazole, dihydrothiodiazole, tetrahydrothiodiazole, tetrahydrooxadiazine, tetrahydrothiadiazine, tetrahydrooxazepine, tetrahydrooxadiazepine, perhydrooxazepine, perhydrooxadiazepine, tetrahydrothiazepine, tetrahydrothiadiazepine, perhydrothiazepine, perhydrothiadiazepine, morpholine, thiomorpholine, indoline, isoindoline, dihydrobenzofuran, perhydrobenzofuran, dihydroisobenzofuran, perhydroisobenzofuran, dihydrobenzothiophene, perhydrobenzothiophene, dihydroisobenzothiophene, perhydroisobenzothiophene, dihydroindazole, perhydroindazole, dihydroquinoline, tetrahydroquinoline, perhydroquinoline, dihydroisoquinoline, tetrahydroisoquinoline, perhydroisoquinoline, dihydrophthalazine, tetrahydrophthalazine, perhydrophthalazine, dihydronaphthyridine, tetrahydronaphthyridine, perhydronaphthyridine, dihydroquinoxaline, tetrahydroquinoxaline, perhydroquinoxaline, dihydroquinazoline, tetrahydroquinazoline, perhydroquinazoline, dihydrocinnoline, tetrahydrocinnoline, perhydrocinnoline, dihydrobenzoxazole, perhydrobenzoxazole, dihydrobenzothiazole, perhydrobenzothiazole, dihydrobenzimidazole, perhydrobenzimidazole, dihydrocarbazole, tetrahydrocarbazole, perhydrocarbazole, dihydroacridine, tetrahydroacridine, perhydroacridine, dihydrodibenzofuran, dihydrodibenzothiophene, tetrahydrodibenzofuran, tetrahydrodibenzothiophene, perhydrodibenzofuran, perhydrodibenzothiophene, dioxolane, dioxane, dithiolane, dithiane, benzodioxalane, benzodioxane, benzodithiolane, benzodithiane, 2,4,6-trioxaspiro[bicyclo[3.3.0]octane-3,1′-cyclohexane], 1,3-dioxolano[4,5-g]chromene or 2-oxabicyclo[2.2.1]heptane ring etc.

C3-8 mono-carbocyclic ring is for example, cyclopropane, cyclobutane, cyclopentane, cyclohexane, cycloheptane, cyclooctane, cyclopentene, cyclohexene, cycloheptene, cyclooctene, cyclopentadiene, cyclohexadiene, cycloheptadiene, cyclooctadiene or benzene ring etc.

The 3-8 membered mono-cyclic hetero ring containing 1-4 nitrogen atom(s), 1-2 oxygen atom(s) and/or 1-2 sulfur atom(s) means 3-8 membered mono-cyclic hetero aryl containing 1-4 nitrogen atom(s), 1-2 oxygen atom and/or 1-2 sulfur atom(s) and partially or fully saturated one.

The 3-8 membered mono-cyclic hetero aryl containing 1-4 nitrogen atom(s), 1-2 oxygen atom(s) and/or 1-2 sulfur atom(s) is pyrrole, imidazole, triazole, tetrazole, pyrazole, pyridine, pyrazine, pyrimidine, pyridazine, azepine, diazepine, furan, pyran, oxepine, thiophene, thiain (thiopyran), thiepine, oxazole, isoxazole, thiazole, isothiazole, furazan, oxadiazole, oxazine, oxadiazine, oxazepine, oxadiazepine, thiadiazole, thiazine, thiadiazine, thiazepine or thiadiazepine ring etc.

In above 3-8 membered mono-cyclic hetero ring containing 1-4 nitrogen atom(s), 1-2 oxygen atom(s) and/or 1-2 sulfur atom(s), partially or fully saturated one is pyrroline, pyrrolidine, imidazoline, imidazolidine, pyrazoline, pyrazolidine, triazoline, triazolidine, tetrazoline, tetrazolidine, dihydropyridine, tetrahydropyridine, piperidine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydroazepine, tetrahydroazepine, perhydroazepine, dihydrodiazepine, tetrahydrodiazepine, perhydrodiazepine, dihydrofuran, tetrahydrofuran, dihydropyran, tetrahydropyran, dihydrothiophene, tetrahydrothiophene, dihydrothiain (dihydrothiopyran), tetrahydrothiain (tetrahydrothiopyran), dihydrooxazole, tetrahydrooxazole, dihydroisoxazole, tetrahydroisoxazole, dihydrothiazole, tetrahydrothiazole, dihydroisothiazole, tetrahydroisothiazole, dihydrooxadiazole, tetrahydrooxadiazole, dihydrothiodiazole, tetrahydrothiodiazole, tetrahydrooxadiazine, tetrahydrothiadiazine, tetrahydrooxazepine, tetrahydrooxadiazepine, perhydrooxazepine, perhydrooxadiazepine, tetrahydrothiazepine, tetrahydrothiadiazepine, perhydrothiazepine, perhydrothiadiazepine, morpholine, thiomorpholine, dioxolane, dioxane, dithiolane or dithiane ring etc.

(i) fused bi-cyclic ring which A ring and B ring bound by two atoms or (ii) spiro ring which A ring and B ring bound by spiro in the formula 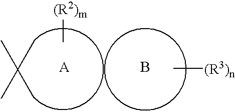
is shown in the following (a) —(ddd).

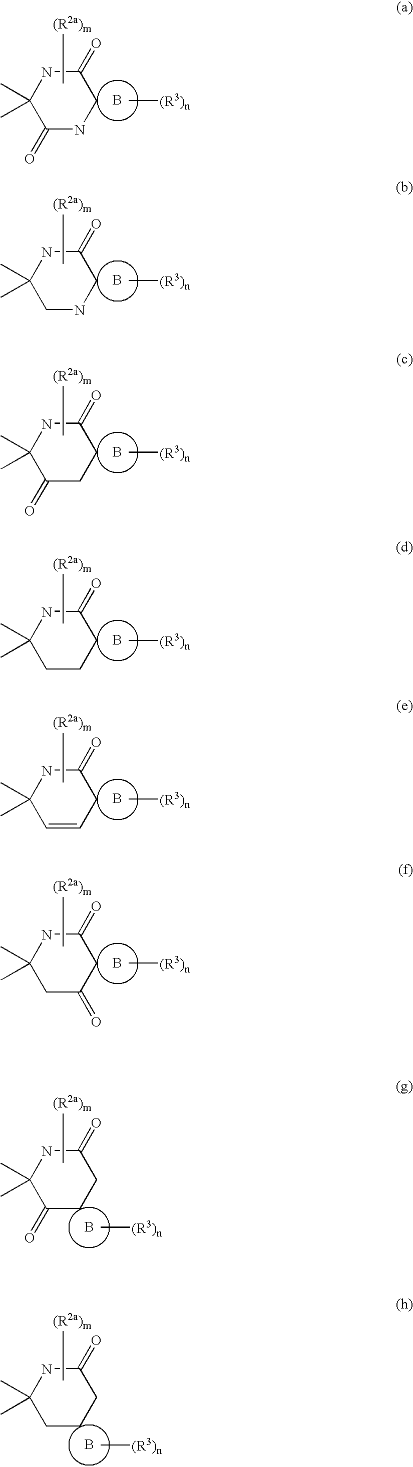

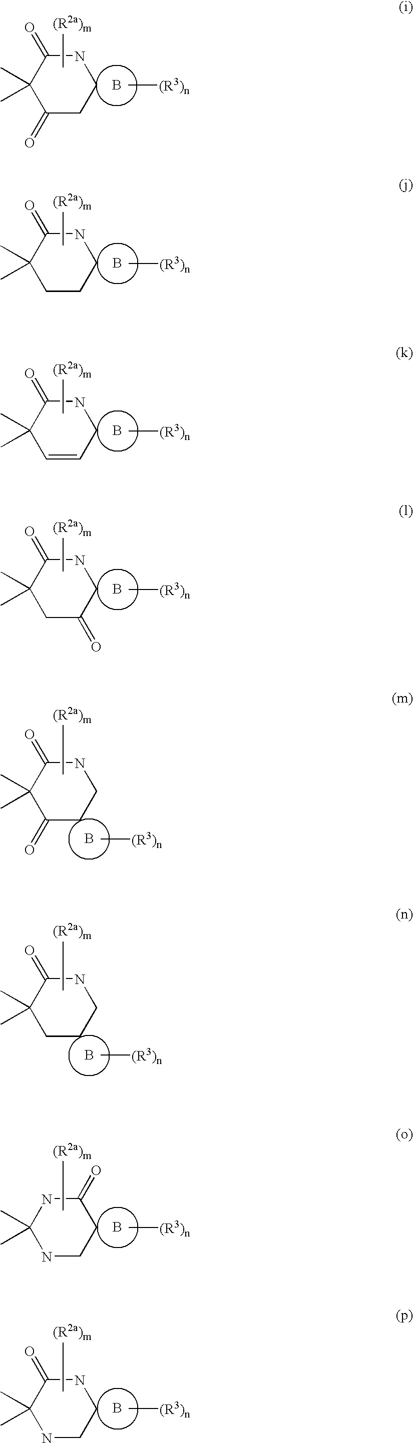

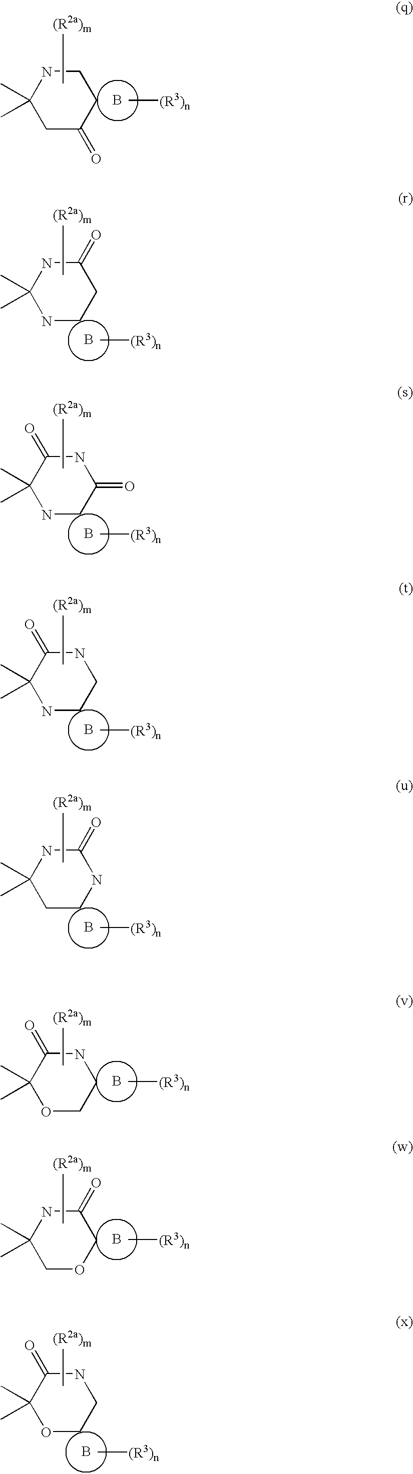

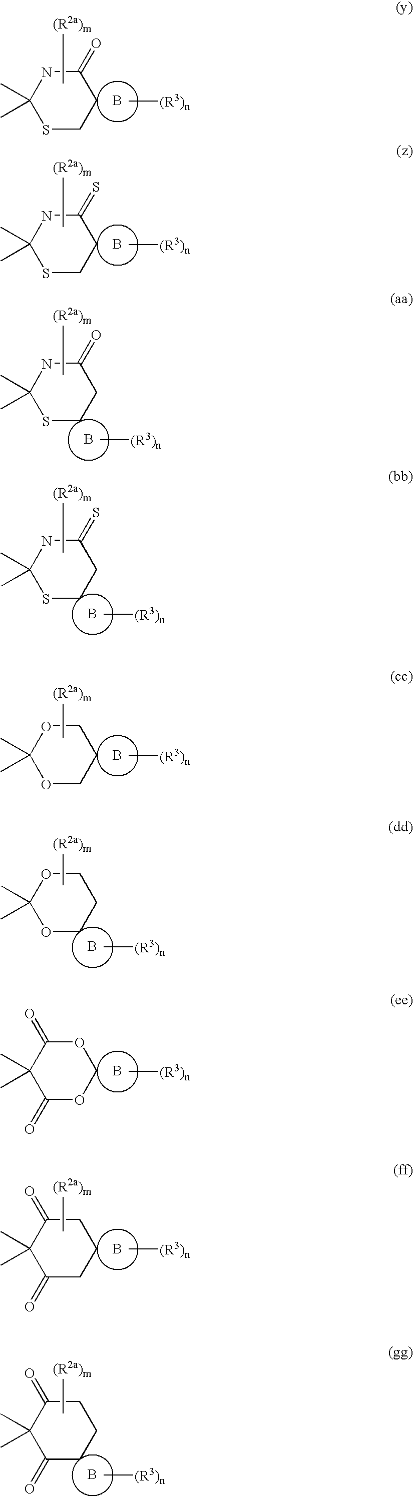

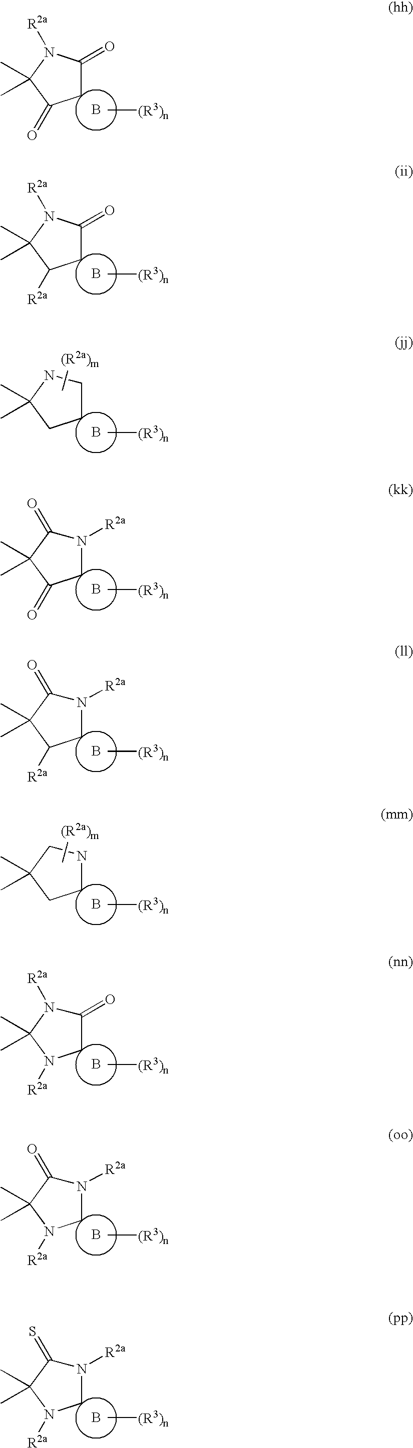

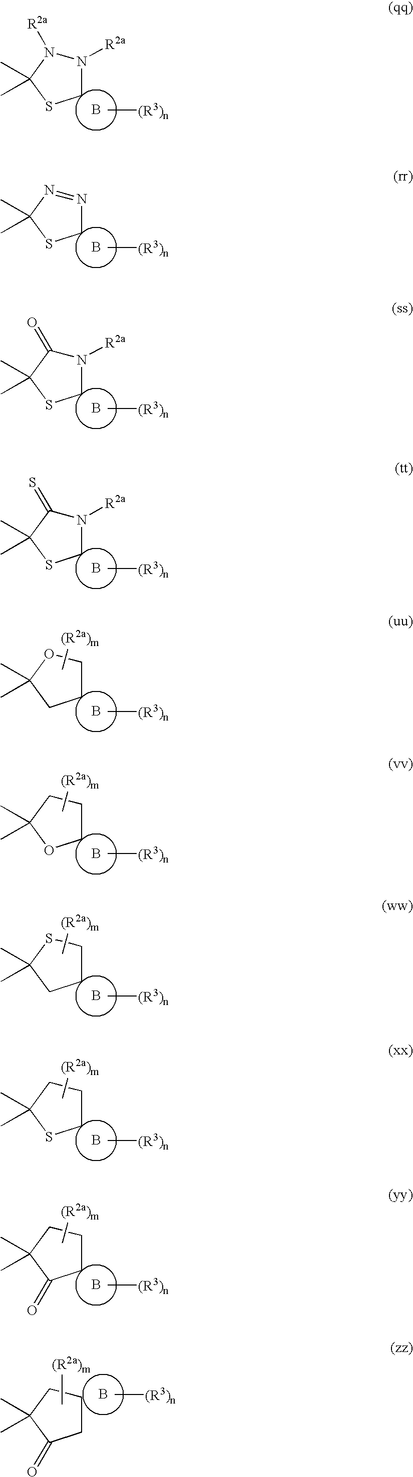

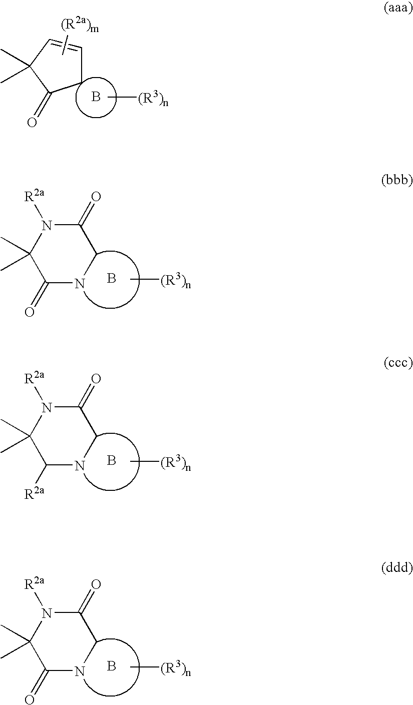

In the above formula, R^{2a} has the same meaning as R², with the proviso that, R²a excludes keto or thioketo, and the other symbols have the same meanings as defined hereinbefore.

C4-7 partially or fully saturated carbocyclic ring represented by B ring means cyclobutane, cyclopentane, cyclohexane, cycloheptane, cyclopentene, cycloheene, cycloheptene, cyclopentadiene, cyclohexadiene, cycloheptadiene ring etc.

The 4-7 membered partially or fully saturated hetero ring containing 1-3 hetero atom(s) selected from a nitrogen atom(s), an oxygen atom(s) and/or a sulfur atom(s) by B ring means pyrroline, pyrrolidine, imidazoline, imidazolidine, pyrazoline, pyrazolidine, triazoline, triazolidine, dihydropyridine, tetrahydropyridine, piperidine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydroazepine, tetrahydroazepine, perhydroazepine, dihydrodiazepine, tetrahydrodiazepine, perhydrodiazepine, dihydrofuran, tetrahydrofuran, dihydropyran, tetrahydropyran, dihydrothiophene, tetrahydrothiophene, dihydrothiain (dihydrothiopyran), tetrahydrothiain (tetrahydrothiopyran), dihydrooxazole, tetrahydrooxazole, dihydroisoxazole, tetrahydroisoxazole, dihydrothiazole, tetrahydrothiazole, dihydroisothiazole, tetrahydroisothiazole, dihydrooxadiazole, tetrahydrooxadiazole, dihydrothiodiazole, tetrahydrothiodiazole, tetrahydrooxadiazine, tetrahydrothiadiazine, tetrahydrooxazepine, tetrahydrooxadiazepine, perhydrooxazepine, perhydrooxadiazepine, tetrahydrothiazepine, tetrahydrothiadiazepine, perhydrothiazepine, perhydrothiadiazepine, morpholine, thiomorpholine, dioxolane, dioxane, dithiolane or dithiane ring etc.

In the present invention, each group represented by R¹, R² or R³ is all preferable.

Preferred R¹ is C1-18 alkyl, C2-18 alkenyl, C2-18 alkynyl, C1-18 alkyl substituted by Cyc 1, C2-18 alkenyl substituted by Cyc 1 or C2-18 alkynyl substituted by Cyc 1, and more preferred R¹ is C1-18 alkyl or C1-6 alkyl substituted by Cyc 1.

Preferred Cyc 1 is a C3-10 mono- or bi-(fused or spiro)carbocyclic ring or a 3-10 membered mono- or bi-(fused or spiro)cyclic hetero ring containing 1-4 nitrogen atom(s), 1-2 oxygen atom(s) and/or 1-2 sulfur atom(s), and more preferred Cyc 1 is a C5-7 mono-carbocyclic aryl or a 5-10 membered mono or bi-cyclic hetero ring containing 1-4 nitrogen atom(s), 2 oxygen atoms and/or 1 sulfur atom.

Preferred Cyc 1 concretely is benzene, pyrazole, imidazole, furan, thiophene, benzodioxane, thiazole or quinoline ring.

Preferred R⁵¹ which is a substituent of Cyc 1, is Cyc 2, —OR⁵²—SR⁵³ or —NR⁵⁴R⁵⁵. Preferred R⁵², R⁵³, R⁵⁴ and R⁵⁵ are C1-8 alkyl or Cyc 2, and preferred C1-8 alkyl is methyl, ethyl, propyl, and preferred Cyc 2 is C5-7 mono-cyclic aryl or 5-7 membered mono-cyclic hetero aryl containing 1-4 nitrogen atom(s), 1 oxygen atom and/or 1 sulfur atom, especially preferred Cyc 2 is benzene.

Preferred R⁷⁷ which is a substituent of Cyc 2 is —COOR⁸¹—CONR⁸³R⁸⁴, —NR¹⁶¹COR¹⁶², —SO₂NR¹⁶³R¹⁶⁴, —NR¹⁶⁶SO₂R¹⁶⁷, C1-8 alkyl substituted by —COOR⁸¹, C1-8 alkyl substituted by —CONR⁸³R⁸⁴, C1-8 alkyl substituted by —NR¹⁶¹C1R¹⁶², C1-8 alkyl substituted by —SO₂NR¹⁶³R¹⁶⁴ or C1-8 alkyl substituted by —NR¹⁶⁶SO₂R¹⁶⁷. Preferred R⁸′, R⁸³, R⁸⁴, R¹⁶¹, R¹⁶², R¹⁶³, R¹⁶⁴ and R¹⁶⁶ are hydrogen, C1-8 alkyl, Cyc 6, C1-8 alkyl substituted by —NR¹⁷⁶R⁷⁷, and more preferred R⁸³, R⁸⁴, R¹⁶¹, R¹⁶², R¹⁶³, R¹⁶⁴, R¹⁶⁶ and R¹⁶⁷ are hydrogen, methyl, ethyl, propyl, phenyl or dimethylaminoethyl etc. Preferred R¹⁶⁷ is C1-8 alkyl, Cyc 6, C1-8 alkyl substituted by —NR¹⁷⁶R¹⁷⁷, and more preferred R¹⁶⁷ is methyl, ethyl, propyl, phenyl or dimethylaminoethyl etc.

Most preferred R¹ is phenylethyl, phenylpropyl, phenylbutyl, phenylpentyl, phenylhexyl, 4-methoxyphenylmethyl, 4-propyloxyphenylmethyl, 4-phenyloxyphenylmethyl, 3,5-dimethyl-1-phenylpyrazol-4-ylmethyl, 2-phenylimidazol-4-ylmethyl, 5-ethylfuran-2-ylmethyl, 5-ethylthiophen-2-ylmethyl, 3-chloro-5-methyl-1-phenylpyrazol-4-ylmethyl, 1,4-benzodioxan-6-ylmethyl, 4-(4-methylsulfonylaminophenyloxy)phenylmethyl, 4-(4-(2-dimethylaminoethylsulfonylamino)phenyloxy)phenylmethyl, 4-(4-dimethylaminosulfonylphenyloxy)phenylmethyl, 4-(4-methylcarbonylaminophenyloxy)phenylmethyl, 4-(4-(2-dimethylaminoethylcarbonylamino)phenyloxy)phenylmethyl, 4-(4-dimethylaminocarbonylphenyloxy)phenylmethyl, or 4-(4-carboxyphenyloxy)phenylmethyl etc.

Preferred R² is keto, thioketo, C1-8 alkyl, C2-8 alkenyl, C2-8 alkynyl, Cyc 3, C1-8 alkyl substituted by Cyc 3 or —OR⁹⁰. Most preferred R² is keto, thioketo, C1-4 alkyl, C2-4 alkenyl or C2-4 alkynyl, Cyc 3, C1-4 alkyl substituted by Cyc 3 or —OR⁹⁰.

Most preferred R² is keto, thioketo, ethyl, propyl, 2-methylpropyl, butyl, 2-propenyl, 2-butenyl, 2-propynyl, 2-butynyl, phenylmethyl, phenylethyl, diphenylethyl, 2-furylmethyl, 2-thienylmethyl, 2-tetrahydrofurylmethyl, 2-tetrahydrothienylmethyl, 1-benzylpyrrolidinyl, 1-benzylpyperidinyl, 2-(indol-3-yl)ethyl, or 2-methoxyethyl etc.

Preferred R³ is hydrogen, C1-8 alkyl, —OR¹²³, —COR¹³¹, —SO₂R¹³³ or C1-8 alkyl substituted by Cyc 4.

Most preferred R³ is methyl, ethyl, propyl, 2-methylpropyl, butyl, hydroxyl, benzyloxy, methoxy, ethoxy, propoxy, 2-methylpropoxy, butoxy, benzyl, cyclopentylmethyl.

Unless otherwise specified, all isomers are included in the present invention. For example, alkyl, alkoxy and alkylene groups include straight or branched ones. In addition, isomers on double bond, ring, fused ring (E-, Z-, cis-, trans-isomer), isomers generated from asymmetric carbon atom(s) (R-, S-, α-, β-isomer, enantiomer, diastereomer), optically active isomer (D-, L-, d-, I-isomer), polar compounds generated by chromatographic separation (more polar compound, less polar compound), equilibrium compounds, mixtures thereof at voluntary ratios and racemic mixtures are also included in the present invention.

Salts:

Non-toxic salts of the present invention include all pharmaceutically acceptable salts, for example, general salts, acid addition salts, hydrate salts.

The compounds of the present invention represented by the formula (I) may be converted into the corresponding salts by conventional means. Water-soluble salts are preferred. Suitable salts, for example, include salts of alkali metals (e.g. potassium, sodium), salts of alkaline earth metals (e.g. calcium, magnesium), ammonium salts, salts of pharmaceutically acceptable organic amines (e.g. tetramethylammonium, triethylamine, methylamine, dimethylamine, cyclopentylamine, benzylamine, phenethylamine, piperidine, monoethanolamine, diethanolamine, tris(hydroxymethyl)amine, lysine, arginine, N-methyl-D-glucamine).

The compounds of the present invention represented by the formula (I) may be converted into the corresponding acid addition salts by conventional means. Water-soluble salts are preferred. Suitable salts, for example, include salts of inorganic acids e.g. hydrochloride, hydrobromide, sulfate, phosphate, nitrate; salts of organic acids e.g. acetate, trifluoroacetate, lactate, tartrate, oxalate, fumarate, maleate, citrate, benzoate, methanesulfonate, ethanesulfonate, benzenesulfonate, toluenesulfonate, isethionate, glucuronate, gluconate.

The compounds of the present invention of the formula (I) and salts thereof may be converted into the corresponding hydrates by conventional means.

All of the compounds of the formula (I) or non-toxic salts thereof are preferable, concretely, the compounds described in the example or non-toxic salts thereof.

Quaternary ammonium salts of the compounds of the formula (I) are the compounds where nitrogen of the compounds represented by the formula (I) is quarternalized by R⁰.

R⁰ is C1-8 alkyl or C1-8 alkyl substituted by phenyl.

N-oxides of the compounds represented by the formula (I) are the compounds where nitrogen of the compounds represented by the formula (I) is oxidized.

Processes for Preparation of the Compounds of the Present Invention:

The compounds of the present invention of the formula (I) may be prepared by the following methods or the methods described in examples.

(1) (a) Among the compounds of the formula (I), the compounds in which R¹ does not represent hydrogen, i.e., the compounds of the formula (I-a) 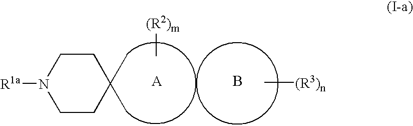
(wherein R^{1a} have the same meaning as R¹, with the proviso that, R^{1a} is not hydrogen and the other symbols have the same meanings as defined hereinbefore)
may be prepared by cyclization of the compounds of the formula (II) 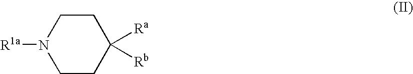
(wherein R^{a} and R^{b} form 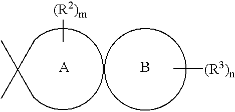
   by cyclization and the other symbols have the same meanings as defined hereinbefore).

This cyclization is well known, for example, it may be carried out by heating in an organic solvent (toluene, dichloromethane or dichloroethane etc.) in the presence or absence of acid (acetic acid, trifluoroacetic acid, hydrochloric acid or p-toluenesulfonic acid etc.) or base (triethylamine, diisopropylethylamine or lithium diisopropylamide etc) at −10 to 120° C.

If necessary, it may be carried out by the conventional method of reduction, oxidation or dehydration reaction in succession to this reaction.

Furthermore, if necessary, the conversion to desired non-toxic salts may be carried out by the conventional method in succession to this reaction.

(b) Among the Compounds of the Formula (I), the Compounds in which R¹ represent hydrogen, i.e., the compounds of the formula (I-b) 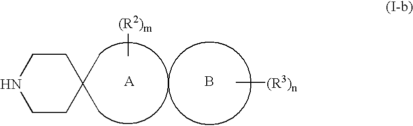
(wherein all symbols have the same meanings as defined hereinbefore)
may be prepared by the removal of an amino protecting group of the compounds in which R^{1a} is an amino-protecting group in the above compound of the formula (I-a).

A protecting group of amino includes, for example, benzyl, benzyloxycarbonyl, allyloxycarbonyl, t-butoxycarbonyl or trifluoroacetyl etc.

The protecting group of amino includes the above one, in addition, the other protecting group which is removable selectively and easily, for example, one described in T. W. Greene et al., Protective Groups in Organic Synthesis, Third Edition, Wiley-Interscience, New York, 1999.

The removal of a protecting group of amino is well known. For example, it is
(1) the alkaline hydrolysis,
(2) the removal of a protecting group in an acidic condition,
(3) the removal of a protecting group by hydrogenolysis, or
(4) the removal of a protecting group using metal complex etc.

Concrete descriptions of these methods are as follows:

(1) The removal of protecting group by alkaline hydrolysis condition (e.g. trifluoroacetyl group) may be carried out, for example, in an organic solvent (methanol, tetrahydrofuran or dioxane etc.) with hydroxide of alkaline metal (sodium hydroxide, potassium hydroxide or lithium hydroxide etc.), hydroxide of alkaline earth metal (barium hydroxide or calcium hydroxide etc.), carbonate (sodium carbonate or potassium carbonate etc.), or an aqueous solution thereof or a mixture thereof at 0-40° C.

(2) The removal of protecting group in an acidic condition (e.g. t-butoxycarbonyl group) may be carried out, for example, in an organic solvent (dichloromethane, chloroform, dioxane, ethyl acetate or anisole etc.), organic acid (acetic acid, trifluoroacetic acid or methanesulfonic acid etc.) or inorganic acid (hydrochloric acid or sulfuric acid etc.), or a mixture thereof (hydrogen bromide/acetic acid etc.) at 0-100C.

(3) The removal of a protecting group by hydrogenolysis (e.g. benzyl, benzyloxycarbonyl or allyloxycarbonyl) may be carried out, for example, in a solvent (ether (tetrahydrofuran, dioxane, dimethoxyethane or diethylether etc.), alcohol (methanol or ethanol etc.), benzene (benzene or toluene etc.), ketone (acetone or methyl ethyl ketone etc.), nitrile (acetonitrile etc.), amide (dimethylformamide etc.), water, ethyl acetate, acetic acid or a mixture thereof etc.) in the presence of a catalyst (palladium on carbon, palladium black, palladium hydroxide on carbon, platinum oxide or Raney nickel etc.), at ordinary or elevated pressure of hydrogen gas or in the presence of ammonium formate at 0-200° C.

(4) The removal of a protecting group using metal complex may be carried out, for example, in an organic solvent (dichloromethane, dimethylformamide or tetrahydrofuran etc.), in the presence of a trap reagent (tributyltin hydride or dimedone etc.) and/or an organic acid (acetic acid, etc.) using a metal complex (tetrakis(triphenylphosphine)palladium (0), etc.) at 0-40° C.

Moreover, the compounds of the formula (I) also may be prepared by the following methods of (c)-(j).

(c) Among the compounds of the formula (I), the compounds, in which R¹ is C1-18 alkyl, C2-18 alkenyl, C2-18 alkynyl, or C1-18 alkyl, C2-18 alkenyl or C2-18 alkynyl substituted by various substituents, and in which R¹ bonds with nitrogen atom through —CH₂—, i.e., the compounds of the formula (I-c) 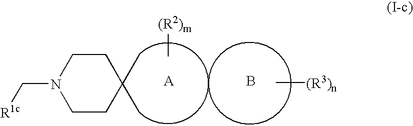
(wherein R^{1C} is C1-17 alkyl, C2-17 alkenyl, C2-17 alkynyl, or C1-17 alkyl, C2-17 alkenyl or C2-17 alkynyl substituted by 1-5 substituent(s) selected from (a) halogen, (b) —CONR⁷R⁸, (c) —COOR⁹, (d) —OR¹⁴, (e) —SR⁵, (f) —NR¹⁶R¹⁷, (g) —NR¹⁸COR¹⁹, (h) —SO₂NR²R²¹, (i) —OCOR²², (j)—NR²³SO₂R²⁴, (k) —NR²⁵COOR²⁶, (l)-NR²⁷CONR²⁸R²⁹ (m) Cyc 1, (n) keto or (O)—N(SO₂R²⁴)₂ and the other symbols have the same meaning as defined hereinbefore)
may be prepared by the reductive amination of the compounds of the formula (I-b) 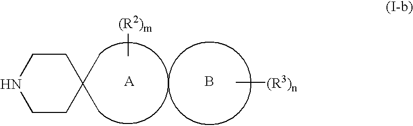
(wherein all symbols have the same meaning as defined hereinbefore) with the compounds the formula (III)
   R^{1C}—CHO (III)
(wherein all symbols have the same meaning as defined hereinbefore).

The reductive amination is well known. For example, it may be carried out in an organic solvent (dichloroethane, dichloromethane, dimethylformamide, acetic acid or a mixture thereof etc.) in the presence of a reducing agent (sodium triacetoxyborohydride or sodium cyanoborohydride etc.) at 0-40° C.

Moreover, the reductive amination may be carried out with the compounds in which nitrogen of R¹ is oxidized to N-oxide.

(d) Among the compounds of the formula (I), the compounds, in which R¹ is C1-18 alkyl, C2-18 alkenyl, C2-18 alkynyl, or C1-18 alkyl, C2-18 alkenyl or C2-18 alkynyl substituted by various substituents, and in which R¹ bonds with nitrogen atom through —CHR^{1d}— (wherein R^{1d} is C1-17 alkyl, C2-17 alkenyl or C2-17 alkynyl.), i.e., the compounds of the formula (I-d) 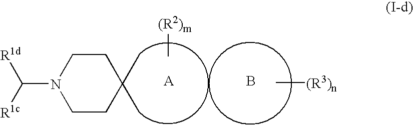
(wherein R^{1d} is C1-17 alkyl, C2-17 alkenyl or C2-17 alkynyl, and the other symbols have the same meanings as defined hereinbefore)
may be prepared by reductive amination of the compounds of the formula (I-b) with the compounds the formula (IV) 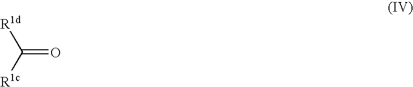
(wherein all of the symbols have the same meanings as defined hereinbefore).

The reductive amination is well known. For example, it may be carried out in an organic solvent (dichloroethane or dichloromethane etc.) in the presence of tertiary amine (triethylamine or diisopropylethylamine etc.) with Lewis acid (titanium tetrachloride etc.), at 0-40° C., and following by the addition of a reducing agent (sodium triacetoxyborohydride or sodium cyanoborohydride etc.) at 0-40° C.

(e) Among the compounds of the formula (I), the compounds in which R¹ is COR⁶, i.e., the compounds of the formula (I-e) 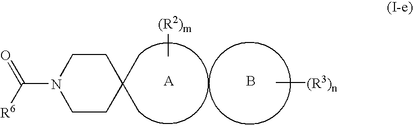
(wherein all of the symbols have the same meanings as defined hereinbefore)
may be prepared by the amidation of the compounds of the formula (I-b) with the compounds of the formula (V) 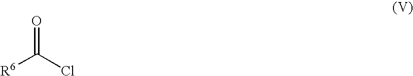
(wherein all of the symbols have the same meanings as defined hereinbefore).

The amidation is well known. For example, it may be carried out in an organic solvent (chloroform, dichloromethane, diethylether, tetrahydrofuran, dioxane or dimethylformamide etc.) in the presence of tertiary amine (isopropylethylamine, pyridine, triethylamine, dimethylaniline or dimethylaminopyridine etc.) or an aqueous alkali solution (aqueous solution of bicarbonate or solution of sodium hydroxide etc.) at 0-40° C.

(f) Among the compounds of the formula (I), the compounds in which R¹ is SO₂R¹⁰, i.e., the compounds of the formula (I-f) 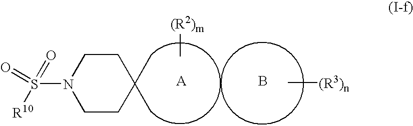
(wherein all of the symbols have the same meanings as defined herein before)
may be prepared by the sulfonamidation of the compounds of the formula (I-b) with the compounds of the formula (VI) 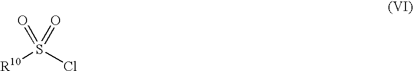
(wherein all of the symbols have the same meanings as defined hereinbefore).

The sulfonamidation is well known. For example, it may be carried out in an inert organic solvent (chloroform, dichloromethane, dichloroethane, diethylether or tetrahydrofuran etc.) in the presence of tertiary amine (diisopropylethylamine, pyridine, triethylamine, dimethylaniline or dimethylaminopyridine etc.) at 0-40° C.

(g) Among the compounds of the formula (I), the compounds in which R¹ is CONR⁷R⁸, i.e., the compounds of the formula (I-g) 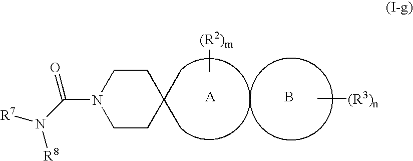
(wherein all of the symbols have the same meanings as defined hereinbefore)
may be prepared by the reaction of the compounds of the formula (I-b) with the compounds of the formula (VII-1) 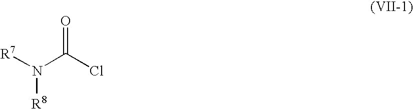
(wherein all of the symbols have the same meanings as defined hereinbefore)
or with the compounds of the formula (VII-2)
   R⁷—N═C═O (VII-2)
(wherein all of the symbols have the same meanings as defined herein before).

The reaction of the compounds of the formula (I-b) with the compounds of the formula (VII-1) is well known. For example, it may be carried out in an organic solvent (chloroform, dichloromethane, diethylether or tetrahydrofuran etc.), in the presence of a tertiary amine (isopropylethylamine, pyridine, triethylamine, dimethylaniline or dimethylaminopyridine etc.) at 0-40° C.

The reaction of the compounds of the formula (I-b) with the compounds of the formula (VII-2) is well known. For example, it may be carried out in an inert organic solvent (chloroform, dichloromethane, dichloroethane, dimethylformamide, diethylether or tetrahydrofuran etc.) at 0-40° C.

(h) Among the compounds of the formula (I), the compounds in which R¹ is —CH₂—CH(OH)—R^{1h} (R^{1h} is C1-16 alkyl, C2-16 alkenyl, C2-16 alkynyl, or C1-16 alkyl, C2-16 alkenyl or C2-16 alkynyl substituted by various substituents.), i.e., the compounds of the formula (I-h) 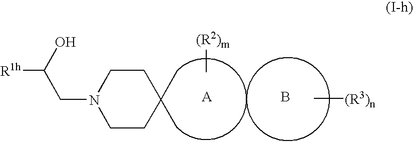
(wherein R^{1h} is C1-16 alkyl, C2-16 alkenyl, C2-16 alkynyl, or C1-16 alkyl, C2-16 alkenyl or C2-16 alkynyl substituted by an substituent(s) selected from (a) halogen, (b) —CONR⁷R⁸, (c) —COOR⁹, (d) —OR¹⁴, (e) —SR⁵, (f) —NR¹⁶R¹⁷, (g) —NR¹⁸COR¹⁹, (h) —SO₂NR²R²¹, (i) —OCOR²², (j)—NR²³SO₂R²⁴, (k) —NR²⁵COOR²⁶, (l) —NR²⁷CONR²⁸R²⁹ (m) Cyc 1, (n) keto or (O)—(SO₂R²⁴)₂, and the other symbols have the same meaning as defined hereinbefore)
may be prepared by the reaction of the compounds of the formula (I-b) with the compounds the formula (VIII) 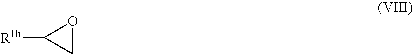
(wherein all of the symbols have the same meanings as defined hereinbefore).

The reaction is well known, and it may be carried out in an organic solvent (methanol, ethanol, 2-propanol, tetrahydrofuran or acetonitrile etc.), in the presence or absence of a tertiary amine (triethylamine or N-methylmorpholine etc.) at 40-100° C.

(j) Among the compounds of the formula (I), the compounds in which R¹ is —CH₂—C(═O)—R^{1j} (R^{1j} has the same meaning as R^{1h}), i.e., the compounds of the formula (I-j) 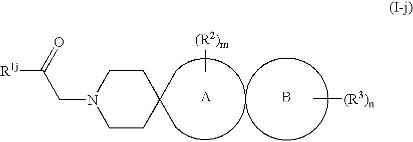
(wherein R^{1j} has the same meaning as R^{1h}, and the other symbols have the same meanings as defined hereinbefore)
may be prepared by the reaction of the compounds of the formula (I-b) with the compounds of the formula (IX-1) 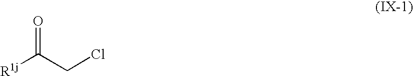
(wherein all of the symbols have the same meanings as defined hereinbefore)
or with the compounds of the formula (IX-2) 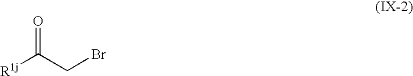
(wherein all of the symbols have the same meanings as defined herein before).

The reaction is well known, and it may be carried out in an organic solvent (chloroform, dichloromethane, diethylether, tetrahydrofuran, dioxane or dimethylformamide etc.) in the presence of a tertiary amine (isopropylethylamine, pyridine, triethylamine, dimethylaniline or dimethylaminopyridine etc.) at 0-40° C.

Among the compounds of the formula (I), the compounds in which at least one group containing carboxyl, hydroxy, amino or thiol may be prepared by the reaction of the above described (a)-(j), and following by the removal of a protecting group using the compounds in which at least one group containing carboxyl, hydroxy, amino and thiol protected by a suitable protecting group.

A protecting group of carboxyl includes, for example, methyl, ethyl, t-butyl, benzyl or allyl.

A protecting group of hydroxy includes, for example, methoxymethyl, 2-tetrahydropyranyl, t-butyldimethylsilyl, t-butyidiphenylsilyl, acetyl or benzyl.

A protecting group of amino includes, for example, benzyloxycarbonyl, allyloxycarbonyl, t-butoxycarbonyl, trifluoroacetyl or 9-fluorenylmethoxycarbonyl.

A protecting group of thiol includes, for example, benzyl, methoxybenzyl, acetoamidomethyl, triphenylmethyl or acetyl.

The protecting group of carboxyl, hydroxy, amino or thiol includes the above one, and in addition the other protecting group which is removable selectively and easily, for example, one described in T. W. Greene et al., Protective Groups in Organic Synthesis, Third Edition, Wiley-Interscience, New York, 1999.

The removal of a protecting group of amino may be carried out by the method described hereinbefore.

The removal of a protecting group of carboxyl, hydroxy or thiol is well known. For example, it is
(1) the alkaline hydrolysis,
(2) the removal of a protecting group in an acidic condition,
(3) the removal of a protecting group by hydrogenolysis, or
(4) the removal of a protecting group silyl or
(5) the removal of a protecting group using metal complex etc.

Among these methods, (1), (2), (3) and (5) may be carried out by the same methods of the removal of a protecting group of amino.

Concretely describing (4), the removal of a protecting group silyl may be carried out, for example, in an organic solvent (tetrahydrofuran or acetonitrile etc.), with tetrabutylammoniumfluoride at 0-40° C.

As well known to the person in the art, the aimed compounds of the present invention may be prepared easily by choice of these removal of a protecting group.

(2) The compounds in which at least one nitrogen is quaternary ammonium salt may be prepared by the reaction of the compounds of the formula (I) with the compounds of the formula (X)
R⁰—Q (X)
(wherein R⁰ is C1-8 alkyl or C1-8 alkyl substituted by phenyl and Q is halogen).

The reaction is well known and it may be carried out, for example, in an organic solvent (acetone, dimethylformamide or methyl ethyl ketone etc.) at 0-40° C.

(3) The compounds in which at least one nitrogen represents N-oxide may be prepared by the oxidation of the compounds of the formula (1).

The oxidation is well known and it may be carried out, for example, in a suitable organic solvent (dichloromethane, chloroform, benzene, hexane or t-butyl alcohol etc.) in the presence of a excessive oxidizing reagent (hydrogen peroxide, sodium periodate, acyl nitrite, sodium perborate, peroxidized acid (for example, 3-chloroperbenzoic acid, peracetic acid etc.), OXONE (brand name, Potassium peroxymonosulfate is abbreviated as OXONE.), potassium permanganate or chromic acid etc.) at 20-60° C.

The compounds of the formula (II), (III), (IV), (V), (VI), (VII-1), (VII-2), (VIII), (IX-1), (IX-2) and (X) have been known per se or may be easily prepared by known methods.

Among the compound of the formula (III), for example, 6-formyl-1,4-benzodioxane is commercially available.

The compound of the formula (II) includes, for example, the compound of the formula (II-1) 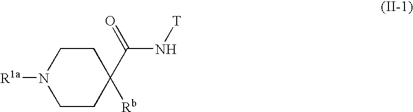
(wherein T is -L-(polystyrene resin), C1-8 alkyl, a C3-8 mono-carbocyclic ring or a C1-8 alkyl substituted by a C3-8 mono-carbocyclic ring or a 5-7 membered heterocyclic ring, the other symbols have the same meanings as defined hereinbefore, and L is a bivalent group)
or the compound of the formula (II-1) 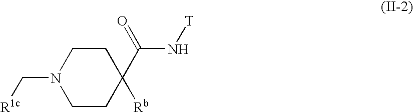
(wherein all symbols have the same meanings as defined hereinbefore).

Bivalent group represented by L in the present invention includes, for example, methylene. Terminal amino polystyrene resin that is NH₂-L-(polystyrene resin) in the present invention includes, for example aminomethylated polystyrene resin.

In the compounds of the formula (II-1) and (II-2), for example, Rb represents 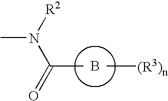
(wherein all symbols have the same meanings as defined hereinbefore.) may be prepared according to the following Scheme 1.

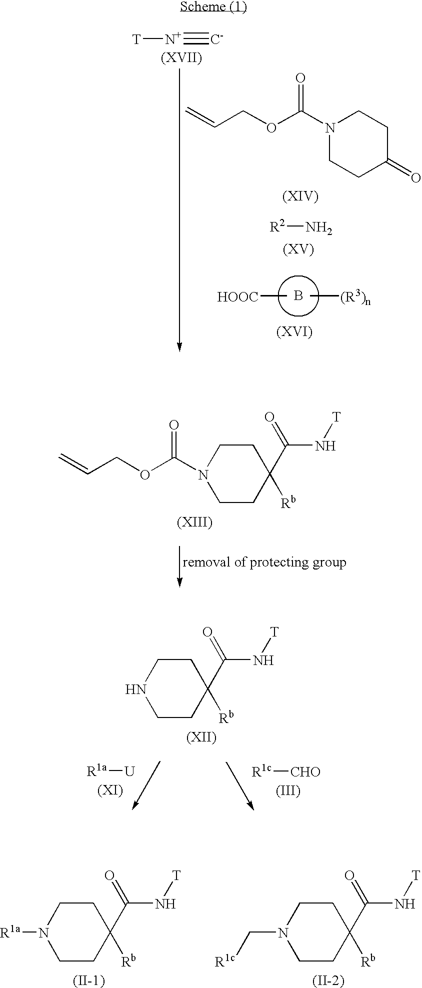

In the scheme (1), U is halogen, and the other symbols have the same meanings as defined hereinbefore.

In the scheme (1), the compound of the formula (XVII) may be commercially available, or prepared by known methods or described methods in example of the following. For example, commercially available compound includes benzylisocyanide, buthylisocyanide, 2-morpholinoethylisocyanide etc.

The other starting materials and each reagent in the present invention have been known per se or may be prepared by known methods.

In the reaction, the reaction products of the present invention may be purified by the conventional methods, for example, washing with a solvent (dimethylformamide, dichloromethane, methanol, tetrahydrofuran, toluene or acetic acid/toluene etc.) at several times. Moreover the obtained products may be purified by conventional techniques. For example, purification may be carried out by distillation at atmospheric or reduced pressure, by high performance liquid chromatography, by thin layer chromatography or by column chromatography using silica gel or magnesium silicate, by washing or by recrystallization.

Pharmacological Activities:

Efficacy of the compounds of the invention represented by general formula (I) was confirmed, e.g., by the following tests.

As described in the foregoing, in order to carry out screening of a compound capable of inhibiting binding of HIV to CXCR4 or CCR5 which is a receptor on the CD4-positive cell, it is a more directive method to carry out it in an assay system that uses HIV virus. However, the use of a large amount of HIV virus in the screening is not practical due to a difficulty in handling it. On the other hand, since both the macrophage tropic (R5) HIV-1 and the ligands, that is, RANTES, MIP-1α and MIP-1β, bind to CCR5, it can be presumed that certain common characteristics are present in the CCR5 binding sites of the HIV side and the RANTES, MIP-1α and MIP-1β sides, and in the binding sides of CCR5 to the ligands, that is, RANTES, MIP-1α and MIP-1β, and HIV. Accordingly, in order to find a compound capable of inhibiting adsorption of HIV virus to cells, which has an inhibitory mechanism different from the current anti-AIDS drugs (reverse transcription inhibitors and protease inhibitors), it is possible to use an assay system that uses an endogeneous CCR5 ligand, RANTES, MIP-1α or MIP-1β instead of HIV.

Specifically, e.g., since CCR5 is a G protein-coupled seven transmembrane type receptor, an assay system in which the effect of RANTES on the transient increase of Ca ion induced via CCR5 is measured can be carried out as an assay system for screening a compound capable of inhibiting binding of RANTES to CCR5. Since both of the T cell tropic (X4) HIV and SDF-1 bind to CXCR4, similar idea can be considered.

Test Methods:

(1) Isolation of Human CCR5 Gene

Human placental cDNA was prepared using Marathon cDNA amplification kit (Clontech). PCR primers hCCR5XbaI-F1:
5′-AGCTAGTCTAGATCCGTTCCCCTACMGAAACTCTCC-3′ (SEQ ID NO:1) and hCCR5XbaI-R1:
5′-AGCTAGTCTAGAGTGCACAACTCTGACTGGGTCACCA-3′ (SEQ ID NO:2) were designed based on the sequence of GenBank U54994.

Using the human placental cDNA as the template and using Ex Taq (Takara), PCR reaction (2 minutes at 95° C.→(30 seconds at 95° C., 45 seconds at 60° C., 1 minute at 72° C.)×35 times) was carried out. The thus amplified PCR product was subjected to a 1% agarose gel electrophoresis, purified using QIAquick Gel Extraction Kit (QUIAGEN) and then digested with a restriction enzyme XbaI. The digested fragments were ligated to an expression vector pEF-BOS-bsr using DNA Ligation Kit Ver. 2 (Takara) and transformed into Escherichia coli DH5a. By preparing the resulting plasmid pEF-BOS-bsr/hCCR5, its DNA sequence was verified.

(2) Culturing of CHO Cell

CHO-dhfr(−) was cultured using Ham's F-12 (containing fetal bovine serum (10%), penicillin (50 U/ml) and streptomycin (50 mg/ml)). Also, the transduced cell was cultured by adding blasticidin (5 mg/ml) to the above medium.

(3) Transduction into CHO Cell

The plasmid pEF-BOS-bsr/hCCR5 was transduced into the CHO-dhfr(−) cell using DMRIE-C reagent (Gibco BRL). After 48 hours, the medium was replaced with a medium containing 5 mg/ml of blasticidin to carry out the selection, thereby establishing a stably over-expressing cell.

(4) Inhibition Test on the Binding of RANTES to CCR5 (Activity of RANTES to Induce Transient Increase of Ca Ion)

The thus established human CCR5 stably over-expressing CHO cell (CCR5/CHO cell) was suspended in Ham's F-12 medium containing FBS (10%) and dispensed in 3.0×10⁶ cells/well portions into a 96 well plate. One day after culturing at 37° C., the culture supernatant was discarded, and Ham's F-12 medium (containing Fura-2AM (5 μM); Probenecid (2.5 mM) and HEPES (20 mM; pH 7.4)) was dispensed in 80 i/well portions to carry out 1 hour of incubation at 37° C. under shaded condition. After washing twice with 1× Hanks/HEPES (20 mM; pH 7.4) solution, the same solution was dispensed in 100 μl/well portions. Each of the test compounds was added to the thus Fura-2AM-incorporated CCR5/CHO cell, and 3 minutes thereafter, a recombinant human RANTES (PeproTach) diluted with 1× Hanks/HEPES (20 mM; pH 7.4) solution was added thereto to a final concentration of 10 nM. Transient increase in the intracellular Ca²⁺ concentration induced by the human RANTES was measured using a Ca²⁺ detector for 96 well use (Hamamatsu Photonics), and inhibition ratio (%) of the test compound was calculated by the following calculation formula.
Inhibition ratio=(Ec−Ea)/Ec×100
Ec: measured value of Ca²⁺ transient increase by RANTES
Ea: measured value of Ca²⁺ transient increase by RANTES when a test compound was added

As a result, the compounds of the invention showed an inhibition ratio of 50% or more at 10 μM. For example, the compound of Example 1(1) showed an IC₅₀ value of 0.74 μM, and the compound of Example 5 an IC₅₀ value of 0.012 μM.

An assay system for finding a compound having adsorption inhibition effect on CCR5 directional HIV strain was described in the foregoing, and it is possible as a matter of course to find a compound capable of inhibiting the activity of CCR5 or a ligand thereof using this system. In the same manner, it is possible to find a compound capable of inhibiting the activity of other chemokine receptor or a ligand thereof.

Toxicity:

The toxicity of the compounds of the present invention is very low and therefore the compounds may be considered safe for pharmaceutical use.

### INDUSTRIAL APPLICABILITY

Application for Pharmaceuticals:

The compounds of the present invention of the formula (I) regulate the effect of chemokine/chemokine receptor in animal included human, especially human, so they are used for prevention and/or treatment of various inflammatory diseases, asthma, atopic dermatitis, urticaria, allergic diseases (allergic bronchopulmonary aspergillosis, allergic eosinophilic gastroenteritis est.), nephritis, nephropathy, hepatitis, arthritis, rheumatoid arthritis, psoriasis, rhinitis, conjunctivitis, ischemic reperfusion disorder, multiple sclerosis, ulcerative colitis, adult respiratory distress syndrome, cytotoxic shock, diabetes, autoimmune disease, multiple organ failure, immunosuppression, cancer metastasis and acquired immune deficiency syndrome.

For the purpose above described, the compounds of formula (I), non-toxic salts thereof, acid addition salts or hydrates thereof may be normally administered systemically or locally, usually by oral or parenteral administration.

The doses to be administered are determined depending upon, for example, age, body weight, symptom, the desired therapeutic effect, the route of administration, and the duration of the treatment. In the human adult, the doses per person are generally from 1 mg to 1000 mg, by oral administration, up to several times per day, and from 1 mg to 100 mg, by parenteral administration (preferably intravenous administration), up to several times per day, or continuous administration from 1 to 24 hours per day from vein.

As mentioned above, the doses to be used depend upon various conditions. Therefore, there are cases in which doses lower than or greater than the ranges specified above may be used.

The compounds of the present invention may be administered in the form of, for example, solid forms for oral administration, liquid forms for oral administration, injections, liniments or suppositories for parenteral administration.

Solid forms for oral administration include compressed tablets, pills, capsules, dispersible powders, and granules. Capsules include hard capsules and soft capsules.

In such solid forms, one or more of the active compound(s) may be admixed with vehicles (such as lactose, mannitol, glucose, microcrystalline cellulose, starch), binders (such as hydroxypropyl cellulose, polyvinylpyrrolidone or magnesium metasilicate aluminate), disintegrants (such as cellulose calcium glycolate), lubricants (such as magnesium stearate), stabilizing agents, and solution adjuvants (such as glutamic acid or aspartic acid) and prepared according to methods well known in normal pharmaceutical practice. The solid forms may, if desired, be coated with coating agents (such as sugar, gelatin, hydroxypropyl cellulose or hydroxypropylmethyl cellulose phthalate), or be coated with two or more films. And further, coating may include containment within capsules of absorbable materials such as gelatin.

Liquid forms for oral administration include pharmaceutically acceptable solutions, suspensions and emulsions, syrups and elixirs. In such forms, one or more of the active compound(s) may be dissolved, suspended or emulized into diluent(s) commonly used in the art (such as purified water, ethanol or a mixture thereof). Besides such liquid forms may also comprise some additives, such as wetting agents, suspending agents, emulsifying agents, sweetening agents, flavoring agents, aroma, preservative or buffering agent.

Injections for parenteral administration include sterile aqueous, suspensions, emulsions and solid forms which are dissolved or suspended into solvent(s) for injection immediately before use. In injections, one or more of the active compound(s) may be dissolved, suspended or emulized into solvent(s). The solvents may include distilled water for injection, physiological salt solution, vegetable oil, propylene glycol, polyethylene glycol, alcohol, e.g. ethanol, or a mixture thereof. Injections may comprise some additives, such as stabilizing agents, solution adjuvants (such as glutamic acid, aspartic acid or POLYSORBATE80 (registered trade mark)), suspending agents, emulsifying agents, soothing agent, buffering agents, preservative. They may be sterilized at a final step, or may be prepared and compensated according to sterile methods. They may also be manufactured in the form of sterile solid forms such as freeze-dried products, which may be dissolved in sterile water or some other sterile diluent(s) for injection immediately before use.

Other forms for parenteral administration include liquids for external use, ointments and endermic liniments, inhalations, sprays, suppositories and pessaries for vaginal administration which comprise one or more of the active compound(s) and may be prepared by methods known per se.

Sprays may comprise additional substances other than diluents, such as stabilizing agents (such as sodium sulfate), isotonic buffers (such as sodium chloride, sodium citrate or citric acid). For preparation of such sprays, for example, the method described in the U.S. Pat. Nos. 2,868,691 or 3,095,355 may be used.

### BEST MODE FOR CARRYING OUT THE INVENTION

The following Reference Examples and Examples are intended to illustrate the present invention, but do not limit them.

In chromatographic separations and TLC, the solvents in parenthesis show the eluting and developing solvents and the ratios of the solvents used are by volume.

The solvents in parenthesis in NMR show the solvents used for measurement.

R* and S* do not represent the absolute position but the relative position.

### REFERENCE EXAMPLE 1

### Preparation of Resin (2)

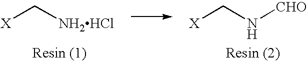

Aminomethylpolystyrene resin hydrochloride (Resin (1); X is polystyrene resin.) (30.0 g) (1% divinylbenzene copolymer, Watanabe Kagaku, Catalog No A00062) was washed with dimethylformamide (300 ml), 10% diisopropylethylamine-dimethylformamide solution (300 ml) and dimethylformamide (300 ml) successively, and was suspended in dimethylformamide (200 ml). To the suspension were added formic acid (10.2 ml) and diisopropylcarbodiimide (42.3 ml) under cooling with ice, and the mixture was stirred for 1 hour at room temperature. The resin was collected by filtration from the reaction mixture, and was washed with dimethylformamide (250 ml×3), dichloromethane (250 ml×4), methanol (250 ml×2) and dichloromethane (250 ml×4) to give Resin (2).

IR (KBr): ν 1682 cm⁻¹.

### REFERENCE EXAMPLE 2

### Preparation of Resin (3)

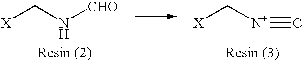

To a suspension of Resin (2) prepared in Reference Example 1 in dichloromethane (300 ml) were added triethylamine (18.8 ml), carbon tetrachloride (13.0 ml) and triphenylphosphine (35.4 g), and the mixture was refluxed for 1 hour. The reaction mixture was cooled to room temperature, and the resin was collected by filtration. The resin was washed with dichloromethane (250 ml×3), methanol (250 ml×1) and dichloromethane (250 ml×2) and dried under reduced pressure to give Resin (3) (28.2 g).

IR (KBr): ν 2147 cm⁻¹.

### REFERENCE EXAMPLE 3

### Preparation of compound (1)

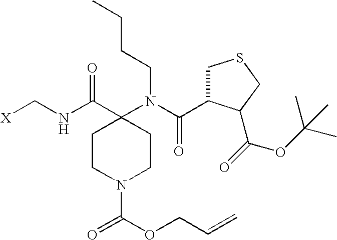

To a suspension of Resin (3) prepared in Reference Example 2 (0.5 g) in tetrahydrofuran/methanol (1:1; 5 ml) were added N-allyloxycarbonyl-4-piperidone (0.40 g), n-butylamine (0.21 ml) and N-(t-butyloxycarbonyl)-L-thioproline (0.51 g), and the mixture was stirred for 16 hours at 65° C. The reaction mixture was cooled to room temperature, and the resin was collected by filtration. The obtained resin was washed with tetrahydrofuran (5 ml×2), methanol (5 ml×2) and dichloromethane (5 ml×2) to give compound (1).

### REFERENCE EXAMPLE 4

### Preparation of compound (2)

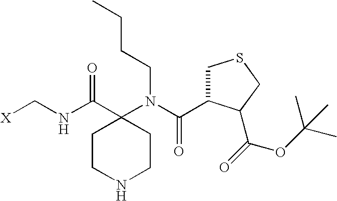

To a suspension of the compound (1) prepared in Reference Example 3 in dichloromethane (5 ml) were added acetic acid (0.16 ml), tributyltin hydride (0.35 ml) and tetrakis(triphenylphosphine)palladium (0) complex (50 mg), and the mixture was stirred for 6 hours at room temperature. The resin was collected by filtration from the reaction mixture, and was washed with dichloromethane (5 ml×3), methanol (5 ml×2), dichloromethane (5 ml×2) and dimethylformamide (5 ml×3) to give compound (2).

### REFERENCE EXAMPLE 5

### Preparation of compound (3)

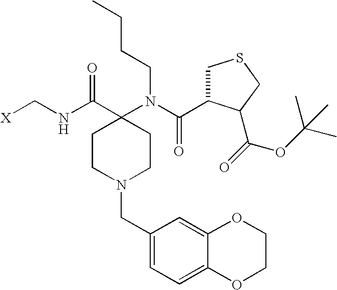

To a suspension of the compound (2) prepared in Reference Example 4 in dimethylformamide (5 ml) were added 6-formyl-1,4-benzodioxane (0.21 g), sodium triacetoxyborohydride (0.28 g) and acetic acid (0.05 ml), and the mixture was stirred for 16 hours at room temperature. The resin was collected by filtration from the reaction mixture, and was washed with dimethylformamide (5 ml×2), dichloromethane (5 ml×2), methanol (5 ml×2) and dichloromethane (5 ml×4) to give compound (3).

### REFERENCE EXAMPLE 6

### Preparation of compound (4)

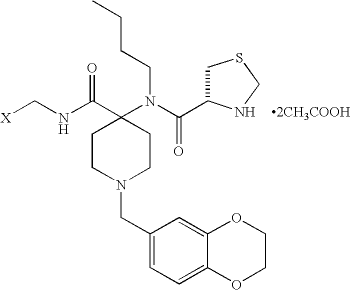

The compound (3) prepared in Reference Example 5 was suspended in 50% trifluoroacetic acid-dichloromethane solution (5 ml), and the suspension was stirred for 5 minutes at room temperature. The reaction mixture was filtrated. The obtained resin was suspended in 50% trifluoroacetic acid-dichloromethane solution (5 ml), and the mixture was stirred for 30 minutes at room temperature. The resin was collected by filtration from the reaction mixture and was washed with dichloromethane (5 ml×4), toluene (5 ml×4), and 1.25 M acetic acid-toluene solution (5 ml×1) to give compound (4).

### EXAMPLE 1

### (3R)-1-butyl-2,5-dioxo-3,4-(2-thiapropano)-9-[(1,4-benzodioxan-6-yl)methyl]-1,4,9-triazaspiro[5.5]undecane hydrochloride

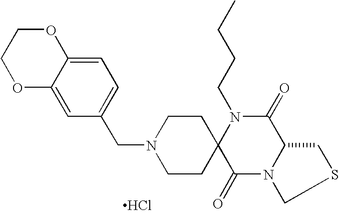

The compound (4) prepared in Reference Example 6 was suspended in 1.25 M acetic acid-toluene solution (5 ml), and the suspension was stirred for 24 hours at 90° C. The reaction mixture was filtrated. The obtained resin was washed with chloroform-methanol (1:1; 5 ml×2). The filtrate and the washings were concentrated. The residue was purified by column chromatography on silica gel (Fuji Silysia Chemical Ltd., FL60D; chloroform:methanol=40:1). A solution of the obtained residue in methanol was acidified by adding 1 N hydrochloric acid, and was concentrated to give the title compound (58 mg) having the following physical data. TLC: Rf 0.56 (chloroform:methanol=10:1);

NMR (CD₃OD): δ 7.08 (d, J=2.2 Hz, 1H), 7.01 (dd, J=8.4, 2.2 Hz, 1H), 6.93 (d, J=8.4 Hz, 1H), 4.88 (d, J=10.2 Hz, 1H), 4.54 (dd, J=9.2, 6.6 Hz, 1H), 4.47 (d, J=10.2 Hz, 1H), 4.27 (s, 4H), 4.17 (s, 2H), 4.08 (m, 1H), 3.70-3.45 (m, 4H), 3.30-3.05 (m, 3H), 2.51 (m, 1H), 2.40-2.15 (m, 3H), 1.80-1.25 (m, 4H), 0.95 (t, J=7.0 Hz, 3H).

### EXAMPLE 1(1)

### (3R)-1-butyl-2,5-dioxo-3,4-(2-thiapropano)-9-[(4-phenoxyphenyl)methyl]-1,4,9-triazaspiro[5.5]undecane hydrochloride

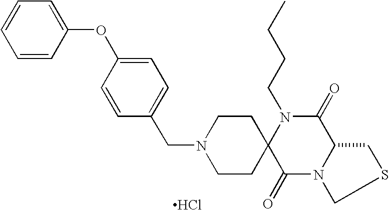

By the same procedure as described in Reference Example 3→Reference Example 4→Reference Example 5→Reference Example 6→Example 1 using the Resin (3) prepared in Reference Example 2, N-allyloxycarbonyl-4-piperidone, n-butylamine, N-(t-butoxycarbonyl)-L-thioproline and 4-phenoxybenzaldehyde, the following compounds of the present invention was obtained. TLC: Rf 0.64 (chloroform:methanol=10:1);

NMR(CD₃OD): δ 7.54 (m, 2H), 7.41 (m, 2H), 7.19 (m, 1H), 7.12-6.98 (m, 4H), 4.89 (d, J=10.4 Hz, 1H), 4.55 (dd, J=9.2, 6.6 Hz, 1H), 4.47 (d, J=10.4 Hz, 1H), 4.33 (s, 2H), 4.11 (m, 1H), 3.70-3.35 (m, 4H), 3.28-3.08 (m, 3H), 2.49 (m, 1H), 2.40-2.18 (m, 3H), 1.75-1.45 (m, 1H), 1.45-1.25 (m, 3H), 0.95 (t, J=7.0 Hz, 3H).

### EXAMPLE 2

### (3S)-1-(2-methylpropyl)-2,5-dioxo-3,4-propano-9-(2-phenylethyl)-1,4,9-triazaspiro[5.5]undecane acetate

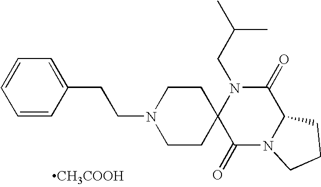

To a suspension of Resin (3) prepared in Reference Example 2 (200 mg) in tetrahydrofuran (1 ml) and methanol (1 ml) were added N-(2-phenylethyl)-4-piperidone (252 mg), isobutylamine (0.123 ml) and N-(t-butyloxycarbonyl)-L-proline (267 mg) at room temperature. The reaction mixture was stirred for 20 hours at 65° C. The reaction mixture was cooled to room temperature and the resin was collected by filtration. The obtained resin was washed with tetrahydrofuran (3 ml×4) and dichloromethane (3 ml×5), and dried. The resin (384 mg) was obtained. To a suspension of the obtained resin (146 mg) in 50% trifluoroacetic acid-dichloromethane solution (2 ml) was stirred for 30 minutes at room temperature. The reaction mixture was filtrated, and the resin was washed with dichloromethane (2 ml×4), methanol (2 ml×4) and dichloromethane (2 ml×4). The obtained resin was suspended in 1.25M acetic acid-toluene solution (2 ml). The reaction mixture was stirred for 20 hours at 90° C. The reaction mixture was filtrated, and the resin was washed with toluene (2 ml×3) and methanol (2 ml×4). The filtrate was concentrated to give the compound of the present invention (7 mg) having the following physical data. TLC: Rf 0.53 (chloroform:methanol=10:1);

NMR(CD₃OD): δ 7.40-7.15 (m, 5H), 4.37 (m, 1H), 3.80-3.40 (m, 5H), 3.20 (m, 1H), 3.10-2.80 (m, 5H), 2.63 (m, 1H), 2.50-2.20 (m, 4H), 2.15-1.75 (m, 5H), 1.98 (s, 3H), 0.94 (d, J=7.0 Hz, 3H), 0.88 (d, J=7.0 Hz, 3H).

### EXAMPLE 2(1)-2(64)

By the same procedure as described in Example 2 using Resin (3) prepared in Reference Example 2, the corresponding 4-piperidone derivatives, the corresponding amine derivatives and the corresponding amino acid derivatives, the following Table 1A-1 to 1A-7, were obtained. Also, compound name and physical data of the above compounds were shown in the following Table 1 B-1 to 1 B-7.

Furthermore, the conditions of high performance liquid chromatography (HPLC) in Tables in the present specification were shown below:

Condition A

Column: YMC-Pack FL-ODS, 50×4.6 mm I.D., S-5 um, 120A
Flow rate: 1 mL/min
Eluent
Component A: 0.1% trifluoroacetic acid aqueous solution
Component B: methanol

The mixture ratio of A and B was fixed in 90/10 for 2 minutes from starting of measurement. The mixture ratio of A and B was linearly changed to 20/80 for 20 minutes. The mixture ratio of A and B was fixed in 20/80 for 5 minutes. The mixture ratio of A and B was linearly changed to 90/10 for 1 minute.

Condition B

Column: YMC-Pack FL-ODS, 50×4.6 mm I.D., S-5 um, 120A
Flow rate: 1 mL/min
Eluent
Component A: 0.1% trifluoroacetic acid aqueous solution
Component B: methanol

The mixture ratio of A and B was fixed in 90/10 for 1 minute from starting of measurement. The mixture ratio of A and B was linearly changed to 10/90 for 16 minutes. The mixture ratio of A and B was fixed in 10/90 for 1 minute. The mixture ratio of A and B was linearly changed to 90/10 for 1 minute.

Condition C

Column: YMC-Pack FL-ODS, 50×4.6 mm I.D., S-5 um, 120A
Flow rate: 1 mL/min
Eluent
Component A: 0.1% trifluoroacetic acid aqueous solution
Component B: methanol

The mixture ratio of A and B was fixed in 90/10 at starting of measurement. The mixture ratio of A and B was linearly changed to 10/90 for 16 minutes. The mixture ratio of A and B was fixed in 10/90 for 0.5 minute. The mixture ratio of A and B was linearly changed to 90/10 for 0.5 minute.

Condition D

Column: YMC-Pack FL-ODS, 50×4.6 mm I.D., S-5 um, 120A
Flow rate: 3 mL/min
Eluent
Component A: 0.1% trifluoroacetic acid aqueous solution
Component B: methanol

The mixture ratio of A and B was fixed in 90/10 at starting of measurement. The mixture ratio of A and B was linearly changed to 10/90 for 5 minutes. The mixture ratio of A and B was fixed in 10/90 for 0.5 minute. The mixture ratio of A and B was linearly changed to 90/10 for 0.1 minute.

Condition E

Column: Xterra™ MS C₁₈ 5 um, 4.6×50 mm I.D.
Flow rate: 3 mL/min
Eluent
Component A: 0.1% trifluoroacetic acid aqueous solution
Component B: 0.1% trifluoroacetic acid-acetonitrile solution

The mixture ratio of A and B was fixed in 95/5 for 0.5 minute from starting of measurement. The mixture ratio of A and B was linearly changed to 0/100 for 2.5 minute. The mixture ratio of A and B was fixed in 0/100 for 0.5 minute. The mixture ratio of A and B was linearly changed to 95/5 for 0.01 minute.

TABLE 1A-1

| Example | Structure |
|---|---|
| 2(1) | 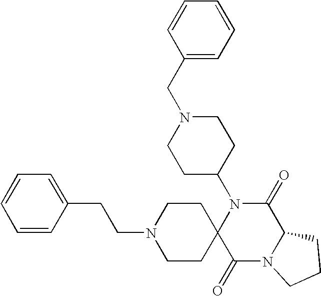 |
| 2(2) | 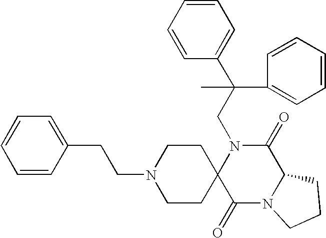 |
| 2(3) | 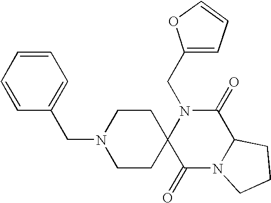 |
| 2(4) | 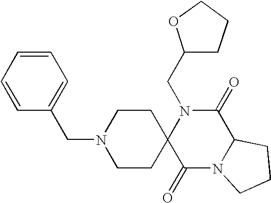 |
| 2(5) | 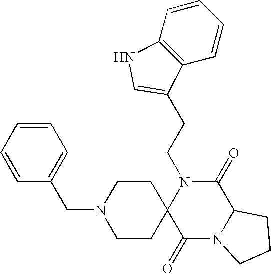 |
| 2(6) | 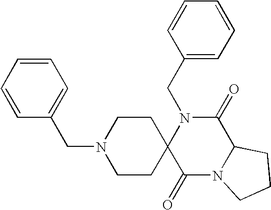 |
| 2(7) | 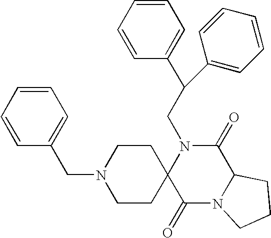 |
| 2(8) | 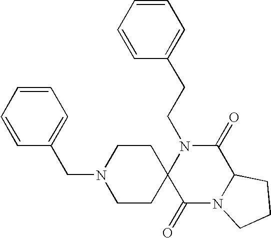 |
| 2(9) | 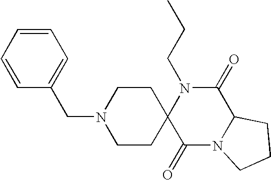 |
| 2(10) | 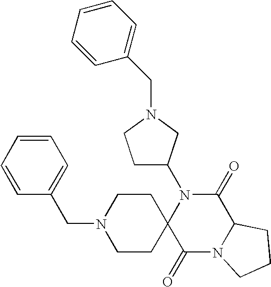 |

TABLE 1A-2

| Example | Structure |
|---|---|
| 2(11) | 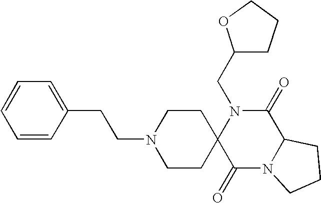 |
| 2(12) | 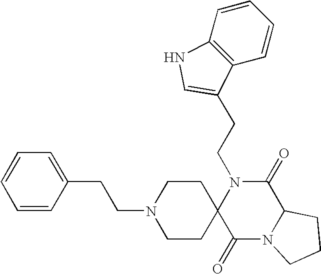 |
| 2(13) | 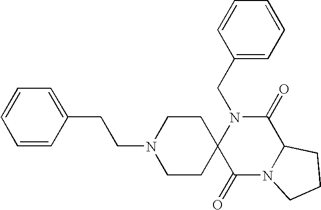 |
| 2(14) | 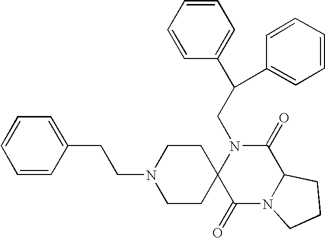 |
| 2(15) | 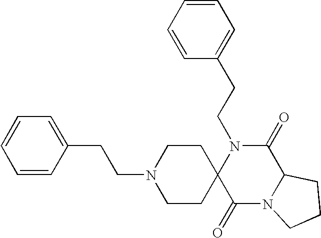 |
| 2(16) | 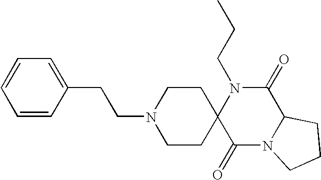 |
| 2(17) | 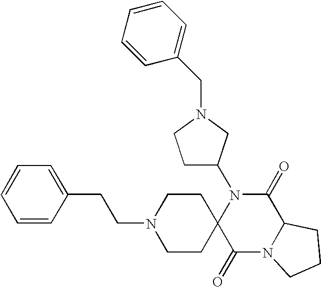 |
| 2(18) | 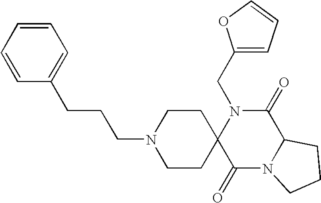 |
| 2(19) | 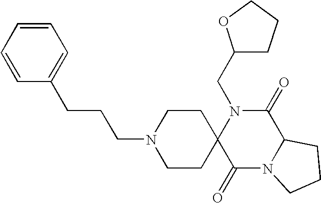 |
| 2(20) | 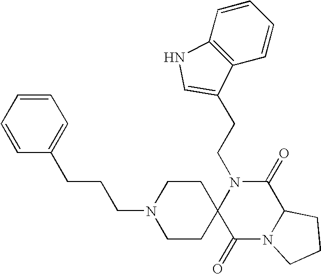 |

TABLE 1A-3

| Example | Structure |
|---|---|
| 2(21) | 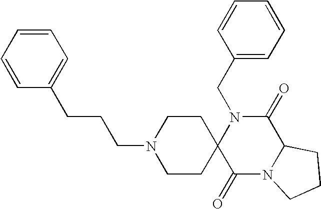 |
| 2(22) | 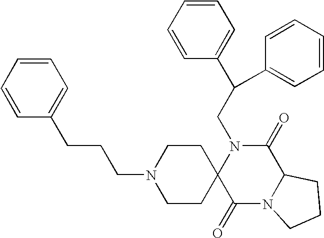 |
| 2(23) | 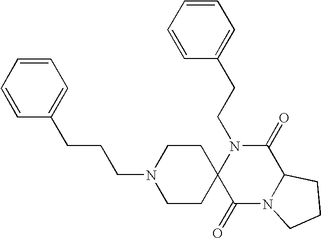 |
| 2(24) | 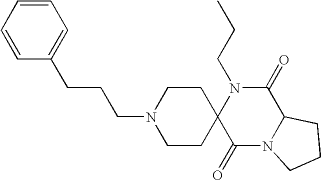 |
| 2(25) | 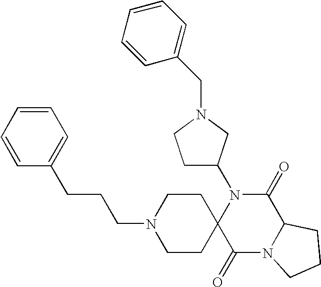 |
| 2(26) | 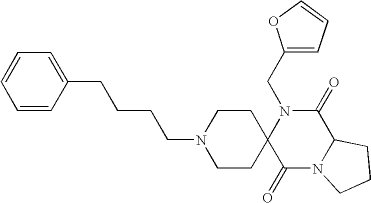 |
| 2(27) | 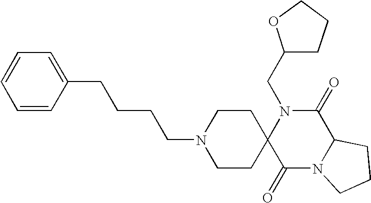 |
| 2(28) | 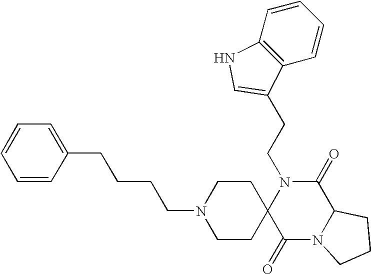 |
| 2(29) | 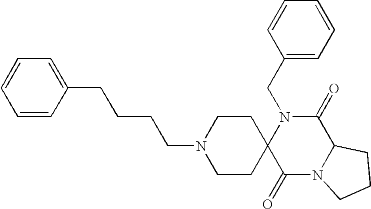 |
| 2(30) | 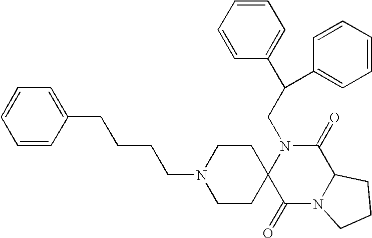 |

TABLE 1A-4

| Example | Structure |
|---|---|
| 2(31) | 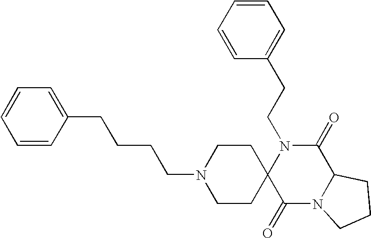 |
| 2(32) | 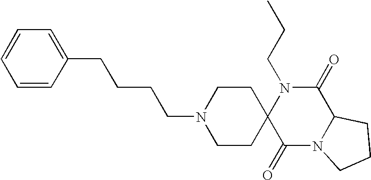 |
| 2(33) | 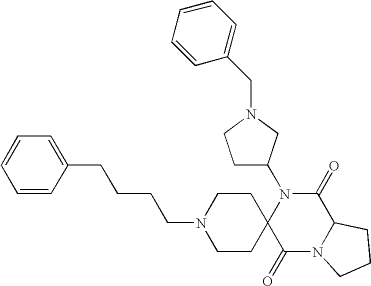 |
| 2(34) | 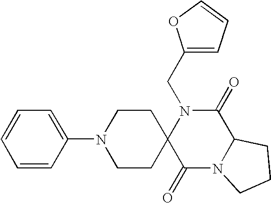 |
| 2(35) | 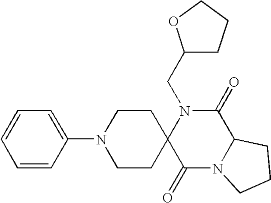 |
| 2(36) | 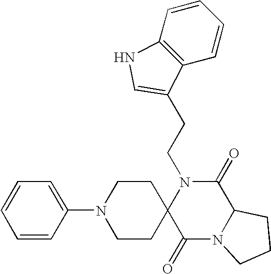 |
| 2(37) | 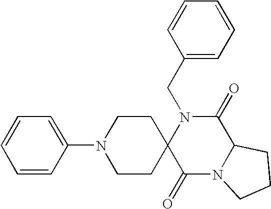 |
| 2(38) | 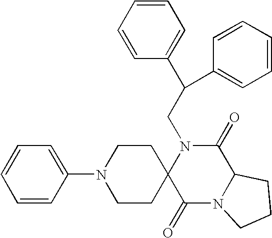 |
| 2(39) | 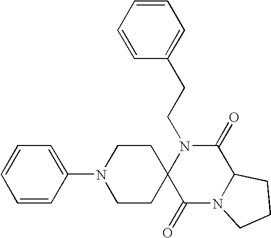 |
| 2(40) | 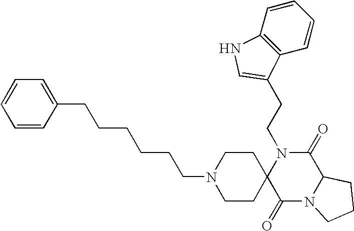 |

TABLE 1A-5

| Example | Structure |
|---|---|
| 2(41) | 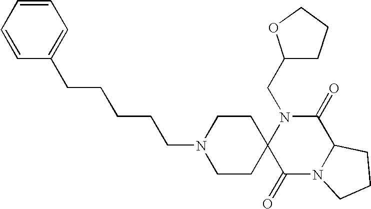 |
| 2(42) | 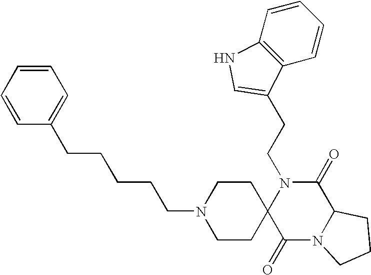 |
| 2(43) | 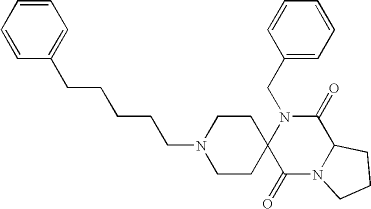 |
| 2(44) | 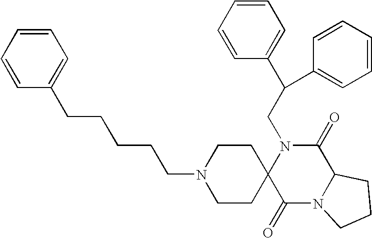 |
| 2(45) | 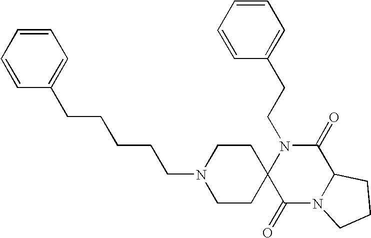 |
| 2(46) | 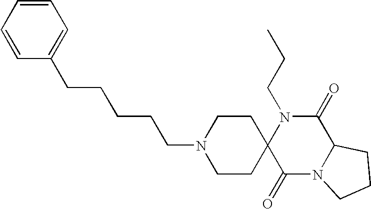 |
| 2(47) | 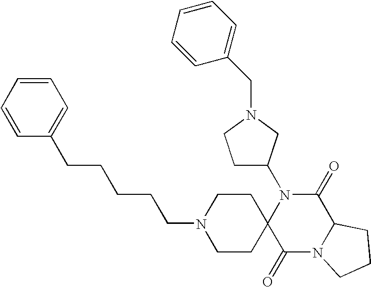 |
| 2(48) | 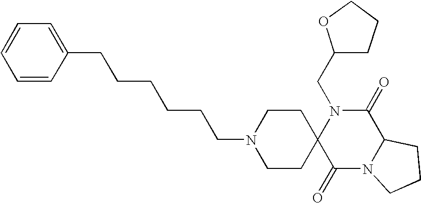 |
| 2(49) | 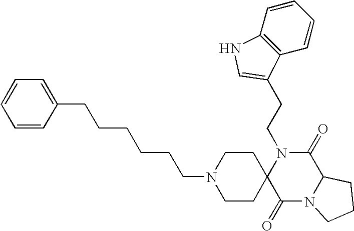 |
| 2(50) | 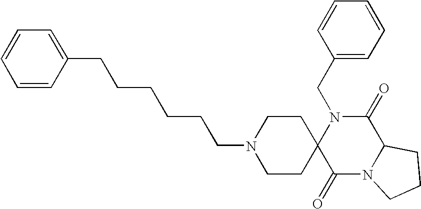 |

TABLE 1A-6

| Example | Structure |
|---|---|
| 2(51) | 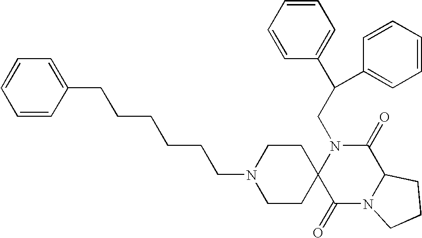 |
| 2(52) | 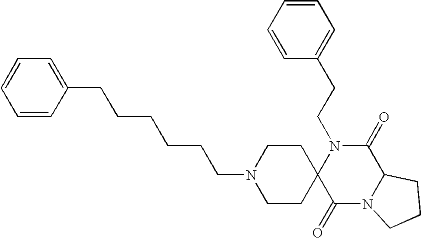 |
| 2(53) | 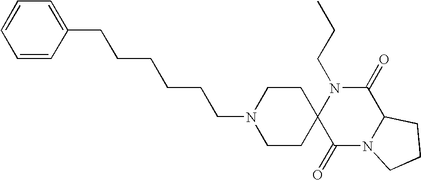 |
| 2(54) | 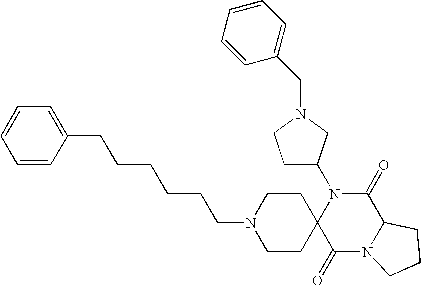 |
| 2(55) | 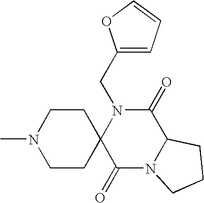 |
| 2(56) | 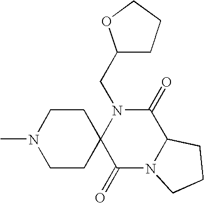 |
| 2(57) | 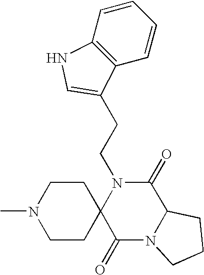 |
| 2(58) | 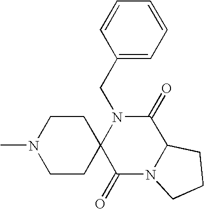 |
| 2(59) | 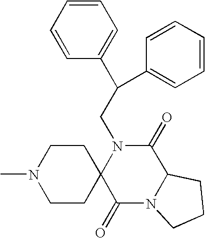 |
| 2(60) | 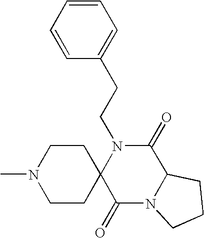 |

TABLE 1A-7

| Example | Structure |
|---|---|
| 2(61) | 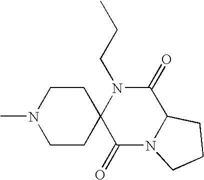 |
| 2(62) | 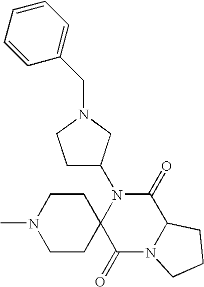 |
| 2(63) | 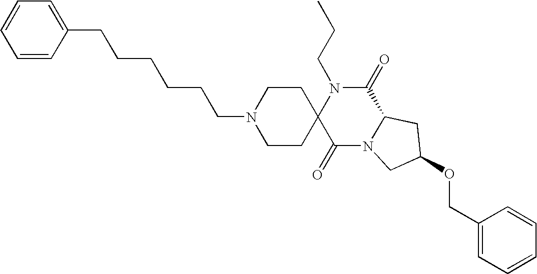 |
| 2(64) | 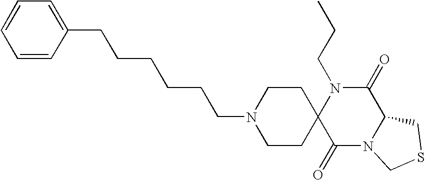 |

TABLE 1B-1

| Example | | HPLC | | Retention | |
|---|---|---|---|---|---|
| Number | Compund Name | condition | Mass condition | time (min) | Mass data |
| 2(1) | (3S)-1-(1-benzyl-4-piperidinyl)- | E | ESI (Pos., 20 V) | 3.11 | 501 (M + H)⁺ |
| | 2,5-dioxo-3,4-propano-9-(2- | | | | |
| | phenylethyl)-1,4,9- | | | | |
| | triazaspiro[5.5]undecane | | | | |
| 2(2) | (3S)-1-(2,2-diphenylpropyl)-2,5- | E | ESI (Pos., 20 V) | 3.66 | 522 (M + H)⁺ |
| | dioxo-3,4-propano-9-(2- | | | | |
| | phenylethyl)-1,4,9- | | | | |
| | triazaspiro[5.5]undecane | | | | |
| 2(3) | 1-(2-furanylmethyl)-2,5-dioxo- | E | ESI (Pos, 20 V) | 3.72 | 394 (M + H)⁺ |
| | 3,4-propano-9-benzyl-1,4,9- | | | | |
| | triazaspiro[5.5]undecane | | | | |
| 2(4) | 1-(2-tetrahydrofuranylmethyl)- | E | ESI (Pos, 20 V) | 2.94 | 398 (M + H)⁺ |
| | 2,5-dioxo-3,4-propano-9-benzyl- | | | | |
| | 1,4,9-triazaspiro[5.5]undecane | | | | |
| 2(5) | 1-(2-(3-indole)ethyl)-2,5-dioxo- | E | ESI (Pos, 20 V) | 3.18 | 457 (M + H)⁺ |
| | 3,4-propano-9-benzyl-1,4,9- | | | | |
| | triazaspiro[5.5]undecane | | | | |
| 2(6) | 1,9-dibenzyl-2,5-dioxo-3,4- | E | ESI (Pos, 20 V) | 3.09 | 404 (M + H)⁺ |
| | propano-1,4,9- | | | | |
| | triazaspiro[5.5]undecane | | | | |
| 2(7) | 1-(2,2-diphenylethyl)-2,5-dioxo- | A | APCI (Pos., 40 V) | 20.7 | 494 (M + H)⁺ |
| | 3,4-propano-9-benzyl-1,4,9- | | | | |
| | triazaspiro[5.5]undecane | | | | |
| 2(8) | 1-(2-phenylethyl)-2,5-dioxo-3,4- | E | ESI (Pos, 20 V) | 3.15 | 418 (M + H)⁺ |
| | propano-9-benzyl-1,4,9- | | | | |
| | triazaspiro[5.5]undecane | | | | |
| 2(9) | 1-propyl-2,5-dioxo-3,4-propano- | E | ESI (Pos, 20 V) | 2.95 | 356 (M + H)⁺ |
| | 9-benzyl-1,4,9- | | | | |
| | triazaspiro[5.5]undecane | | | | |
| 2(10) | 1-(1-benzyl-3-pyrrolidinyl)-2,5- | E | ESI (Pos, 20 V) | 2.93 | 473 (M + H)⁺ |
| | dioxo-3,4-propano-9-benzyl- | | | | |
| | 1,4,9-triazaspiro[5.5]undecane | | | | |

TABLE 1B-2

| Example | | HPLC | | Retention | |
|---|---|---|---|---|---|
| Number | Compund Name | condition | Mass condition | time (min) | Mass data |
| 2(11) | 1-(2-tetrahydrofuranylmethyl)-2,5- | E | ESI (Pos, 20 V) | 3.01 | 412 (M + H)⁺ |
| | dioxo-3,4-propano-9-(2- | | | | |
| | phenylethyl)-1,4,9- | | | | |
| | triazaspiro[5.5]undecane | | | | |
| 2(12) | 1-(2-(3-indole)ethyl)-2,5-dioxo- | E | ESI (Pos, 20 V) | 3.24 | 471 (M + H)⁺ |
| | 3,4-propano-9-(2-phenylethyl)- | | | | |
| | 1,4,9-triazaspiro[5.5]undecane | | | | |
| 2(13) | 1-benzyl-2,5-dioxo-3,4-propano-9- | E | ESI (Pos, 20 V) | 3.16 | 418 (M + H)⁺ |
| | (2-phenylethyl)-1,4,9- | | | | |
| | triazaspiro[5.5]undecane | | | | |
| 2(14) | 1-(2,2-diphenylethyl)-2,5-dioxo- | E | ESI (Pos, 20 V) | 3.4 | 508 (M + H)⁺ |
| | 3,4-propano-9-(2-phenylethyl)- | | | | |
| | 1,4,9-triazaspiro[5.5]undecane | | | | |
| 2(15) | 1-(2-phenylethyl)-2,5-dioxo-3,4- | E | ESI (Pos, 20 V) | 3.22 | 432 (M + H)⁺ |
| | propano-9-(2-phenylethyl)-1,4,9- | | | | |
| | triazaspiro[5.5]undecane | | | | |
| 2(16) | 1-propyl-2,5-dioxo-3,4-propano-9- | E | ESI (Pos, 20 V) | 3.02 | 370 (M + H)⁺ |
| | (2-phenylethyl)-1,4,9- | | | | |
| | triazaspiro[5.5]undecane | | | | |
| 2(17) | 1-(1-benzyl-3-pyrrolidinyl)-2,5- | E | ESI (Pos, 20 V) | 2.97 | 487 (M + H)⁺ |
| | dioxo-3,4-propano-9-(2- | | | | |
| | phenylethyl)-1,4,9- | | | | |
| | triazaspiro[5.5]undecane | | | | |
| 2(18) | 1-(2-furanylmethyl)-2,5-dioxo-3,4- | E | ESI (Pos, 20 V) | 3.13 | 422 (M + H)⁺ |
| | propano-9-(3-phenylpropyl)-1,4,9- | | | | |
| | triazaspiro[5.5]undecane | | | | |
| 2(19) | 1-(2-tetrahydrofuranylmethyl)-2,5- | E | ESI (Pos, 20 V) | 3.07 | 426 (M + H)⁺ |
| | dioxo-3,4-propano-9-(3- | | | | |
| | phenylpropyl)-1,4,9- | | | | |
| | triazaspiro[5.5]undecane | | | | |
| 2(20) | 1-(2-(3-indole)ethyl)-2,5-dioxo- | E | ESI (Pos, 20 V) | 3.29 | 485 (M + H)⁺ |
| | 3,4-propano-9-(3-phenylpropyl)- | | | | |
| | 1,4,9-triazaspiro[5.5]undecane | | | | |

TABLE 1B-3

| Example | | HPLC | | Retention | |
|---|---|---|---|---|---|
| Number | Compund Name | condition | Mass condition | time (min) | Mass data |
| 2(21) | 1-benzyl-2,5-dioxo-3,4-propano- | E | ESI (Pos, 20 V) | 3.22 | 432 (M + H)⁺ |
| | 9-(3-phenylpropyl)-1,4,9- | | | | |
| | triazaspiro[5.5]undecane | | | | |
| 2(22) | 1-(2,2-diphenylethyl)-2,5-dioxo- | E | ESI (Pos, 20 V) | 3.46 | 522 (M + H)⁺ |
| | 3,4-propano-9-(3-phenylpropyl)- | | | | |
| | 1,4,9-triazaspiro[5.5]undecane | | | | |
| 2(23) | 1-(2-phenylethyl)-2,5-dioxo-3,4- | E | ESI (Pos, 20 V) | 3.28 | 446 (M + H)⁺ |
| | propano-9-(3-phenylpropyl)- | | | | |
| | 1,4,9-triazaspiro[5.5]undecane | | | | |
| 2(24) | 1-propyl-2,5-dioxo-3,4-propano- | E | ESI (Pos, 20 V) | 3.09 | 384 (M + H)⁺ |
| | 9-(3-phenylpropyl)-1,4,9- | | | | |
| | triazaspiro[5.5]undecane | | | | |
| 2(25) | 1-(1-benzyl-3-pyrrolidinyl)-2,5- | E | ESI (Pos, 20 V) | 3.03 | 501 (M + H)⁺ |
| | dioxo-3,4-propano-9-(3- | | | | |
| | phenylpropyl)-1,4,9- | | | | |
| | triazaspiro[5.5]undecane | | | | |
| 2(26) | 1-(2-furanylmethyl)-2,5-dioxo- | E | ESI (Pos, 20 V) | 3.22 | 436 (M + H)⁺ |
| | 3,4-propano-9-(4-phenylbutyl)- | | | | |
| | 1,4,9-triazaspiro[5.5]undecane | | | | |
| 2(27) | 1-(2-tetrahydrofuranylmethyl)- | E | ESI (Pos, 20 V) | 3.14 | 440 (M + H)⁺ |
| | 2,5-dioxo-3,4-propano-9-(4- | | | | |
| | phenylbutyl)-1,4,9- | | | | |
| | triazaspiro[5.5]undecane | | | | |
| 2(28) | 1-(2-(3-indole)ethyl)-2,5-dioxo- | E | ESI (Pos, 20 V) | 3.35 | 499 (M + H)⁺ |
| | 3,4-propano-9-(4-phenylbutyl)- | | | | |
| | 1,4,9-triazaspiro[5.5]undecane | | | | |
| 2(29) | 1-benzyl-2,5-dioxo-3,4-propano- | E | ESI (Pos, 20 V) | 3.29 | 446 (M + H)⁺ |
| | 9-(4-phenylbutyl)-1,4,9- | | | | |
| | triazaspiro[5.5]undecane | | | | |
| 2(30) | 1-(2,2-diphenylethyl)-2,5-dioxo- | E | ESI (Pos, 20 V) | 3.51 | 536 (M + H)⁺ |
| | 3,4-propano-9-(4-phenylbutyl)- | | | | |
| | 1,4,9-triazaspiro[5.5]undecane | | | | |

TABLE 1B-4

| Example | | HPLC | | Retention | |
|---|---|---|---|---|---|
| Number | Compund Name | condition | Mass condition | time (min) | Mass data |
| 2(31) | 1-(2-phenylethyl)-2,5-dioxo- | E | ESI (Pos, 20 V) | 3.34 | 460 (M + H)⁺ |
| | 3,4-propano-9-(4-phenylbutyl)- | | | | |
| | 1,4,9-triazaspiro[5.5]undecane | | | | |
| 2(32) | 1-propyl-2,5-dioxo-3,4- | E | ESI (Pos, 20 V) | 3.17 | 398 (M + H)⁺ |
| | propano-9-(4-phenylbutyl)- | | | | |
| | 1,4,9-triazaspiro[5.5]undecane | | | | |
| 2(33) | 1-(1-benzyl-3-pyrrolidinyl)-2,5- | E | ESI (Pos, 20 V) | 3.09 | 515 (M + H)⁺ |
| | dioxo-3,4-propano-9-(4- | | | | |
| | phenylbutyl)-1,4,9- | | | | |
| | triazaspiro[5.5]undecane | | | | |
| 2(34) | 1-(2-furanylmethyl)-2,5-dioxo- | E | ESI (Pos, 20 V) | 3.02 | 380 (M + H)⁺ |
| | 3,4-propano-9-phenyl-1,4,9- | | | | |
| | triazaspiro[5.5]undecane | | | | |
| 2(35) | 1-(2-tetrahydrofuranylmethyl)- | E | ESI (Pos, 20 V) | 2.91 | 384 (M + H)⁺ |
| | 2,5-dioxo-3,4-propano-9- | | | | |
| | phenyl-1,4,9- | | | | |
| | triazaspiro[5.5]undecane | | | | |
| 2(36) | 1-(2-(3-indole)ethyl)-2,5-dioxo- | E | ESI (Pos, 20 V) | 3.18 | 443 (M + H)⁺ |
| | 3,4-propano-9-phenyl-1,4,9- | | | | |
| | triazaspiro[5.5]undecane | | | | |
| 2(37) | 1-benzyl-2,5-dioxo-3,4- | E | ESI (Pos, 20 V) | 3.13 | 390 (M + H)⁺ |
| | propano-9-phenyl-1,4,9- | | | | |
| | triazaspiro[5.5]undecane | | | | |
| 2(38) | 1-(2,2-diphenylethyl)-2,5- | E | ESI (Pos, 20 V) | 3.42 | 480 (M + H)⁺ |
| | dioxo-3,4-propano-9-phenyl- | | | | |
| | 1,4,9-triazaspiro[5.5]undecane | | | | |
| 2(39) | 1-(2-phenylethyl)-2,5-dioxo- | E | ESI (Pos, 20 V) | 3.18 | 404 (M + H)⁺ |
| | 3,4-propano-9-phenyl-1,4,9- | | | | |
| | triazaspiro[5.5]undecane | | | | |
| 2(40) | 1-propyl-2,5-dioxo-3,4- | E | ESI (Pos, 20 V) | 2.92 | 342 (M + H)⁺ |
| | propano-9-phenyl-1,4,9- | | | | |
| | triazaspiro[5.5]undecane | | | | |

TABLE 1B-5

| Example | | HPLC | | Retention | |
|---|---|---|---|---|---|
| Number | Compund Name | condition | Mass condition | time (min) | Mass data |
| 2(41) | 1-(2-tetrahydrofuranylmethyl)- | C | APCI (Pos., 40 V) | 13.3 | 454 (M + H)⁺ |
| | 2,5-dioxo-3,4-propano-9-(5- | | | | |
| | phenylpentyl)-1,4,9- | | | | |
| | triazaspiro[5.5]undecane | | | | |
| 2(42) | 1-(2-(3-indole)ethyl)-2,5- | E | ESI (Pos., 20 V) | 3.48 | 513 (M + H)⁺ |
| | dioxo-3,4-propano-9-(5- | | | | |
| | phenylpentyl)-1,4,9- | | | | |
| | triazaspiro[5.5]undecane | | | | |
| 2(43) | 1-benzyl-2,5-dioxo-3,4- | E | ESI (Pos, 20 V) | 3.42 | 460 (M + H)⁺ |
| | propano-9-(5-phenylpentyl)- | | | | |
| | 1,4,9- | | | | |
| | triazaspiro[5.5]undecane | | | | |
| 2(44) | 1-(2,2-diphenylethyl)-2,5- | B | APCI (Pos., 40 V) | 17.0 | 550 (M + H)⁺, |
| | dioxo-3,4-propano-9-(5- | | | | 400, 293, 181 |
| | phenylpentyl)-1,4,9- | | | | |
| | triazaspiro[5.5]undecane | | | | |
| 2(45) | 1-(2-phenylethyl)-2,5-dioxo- | E | ESI (Pos, 20 V) | 3.47 | 474 (M + H)⁺ |
| | 3,4-propano-9-(5- | | | | |
| | phenylpentyl)-1,4,9- | | | | |
| | triazaspiro[5.5]undecane | | | | |
| 2(46) | 1-propyl-2,5-dioxo-3,4- | E | ESI (Pos, 20 V) | 3.3 | 412 (M + H)⁺ |
| | propano-9-(5-phenylpentyl)- | | | | |
| | 1,4,9- | | | | |
| | triazaspiro[5.5]undecane | | | | |
| 2(47) | 1-(1-benzyl-3-pyrrolidinyl)- | B | APCI (Pos., 40 V) | 13.9 | 529 (M + H)⁺, |
| | 2,5-dioxo-3,4-propano-9-(5- | | | | 464, 370, 220 |
| | phenylpentyl)-1,4,9- | | | | |
| | triazaspiro[5.5]undecane | | | | |
| 2(48) | 1-(2-tetrahydrofuranylmethyl)- | E | ESI (Pos., 20 V) | 3.38 | 468 (M + H)⁺ |
| | 2,5-dioxo-3,4-propano-9-(6- | | | | |
| | phenylhexyl)-1,4,9- | | | | |
| | triazaspiro[5.5]undecane | | | | |
| 2(49) | 1-(2-(3-indole)ethyl)-2,5- | E | ESI (Pos., 20 V) | 3.55 | 527 (M + H)⁺ |
| | dioxo-3,4-propano-9-(6- | | | | |
| | phenylhexyl)-1,4,9- | | | | |
| | triazaspiro[5.5]undecane | | | | |
| 2(50) | 1-benzyl-2,5-dioxo-3,4- | B | APCI (Pos., 40 V) | 16.5 | 474 (M + H)⁺, |
| | propano-9-(6-phenylhexyl)- | | | | 310, 201 |
| | 1,4,9- | | | | |
| | triazaspiro[5.5]undecane | | | | |

TABLE 1B-6

| Example | | HPLC | | Retention | |
|---|---|---|---|---|---|
| Number | Compund Name | condition | Mass condition | time (min) | Mass data |
| 2(51) | 1-(2,2-diphenylethyl)-2,5- | E | ESI (Pos., 20 V) | 3.7 | 564 (M + H)⁺ |
| | dioxo-3,4-propano-9-(6- | | | | |
| | phenylhexyl)-1,4,9- | | | | |
| | triazaspiro[5.5]undecane | | | | |
| 2(52) | 1-(2-phenylethyl)-2,5-dioxo- | B | APCI (Pos., 40 V) | 16.9 | 488 (M + H)⁺, |
| | 3,4-propano-9-(6- | | | | 324, 217 |
| | phenylhexyl)-1,4,9- | | | | |
| | triazaspiro[5.5]undecane | | | | |
| 2(53) | 1-propyl-2,5-dioxo-3,4- | C | APCI (Pos., 40 V) | 14.0 | 426 (M + H)⁺, |
| | propano-9-(6-phenylhexyl)- | | | | 262 |
| | 1,4,9- | | | | |
| | triazaspiro[5.5]undecane | | | | |
| 2(54) | 1-(1-benzyl-3-pyrrolidinyl)-2,5- | E | ESI (Pos., 20 V) | 3.3 | 543 (M + H)⁺ |
| | dioxo-3,4-propano-9-(6- | | | | |
| | phenylhexyl)-1,4,9- | | | | |
| | triazaspiro[5.5]undecane | | | | |
| 2(55) | 1-(2-furanylmethyl)-2,5-dioxo- | E | ESI (Pos., 20 V) | 2.72 | 318 (M + H)⁺ |
| | 3,4-propano-9-methyl-1,4,9- | | | | |
| | triazaspiro[5.5]undecane | | | | |
| 2(56) | 1-(2-tetrahydrofuranylmethyl)- | E | ESI (Pos., 20 V) | 2.59 | 322 (M + H)⁺ |
| | 2,5-dioxo-3,4-propano-9- | | | | |
| | methyl-1,4,9- | | | | |
| | triazaspiro[5.5]undecane | | | | |
| 2(57) | 1-(2-(3-indole)ethyl)-2,5- | E | ESI (Pos., 20 V) | 2.96 | 381 (M + H)⁺ |
| | dioxo-3,4-propano-9-methyl- | | | | |
| | 1,4,9- | | | | |
| | triazaspiro[5.5]undecane | | | | |
| 2(58) | 1-benzyl-2,5-dioxo-3,4- | E | ESI (Pos., 20 V) | 2.87 | 328 (M + H)⁺ |
| | propano-9-methyl-1,4,9- | | | | |
| | triazaspiro[5.5]undecane | | | | |
| 2(59) | 1-(2,2-diphenylethyl)-2,5- | E | ESI (Pos., 20 V) | 3.16 | 418 (M + H)⁺ |
| | dioxo-3,4-propano-9-methyl- | | | | |
| | 1,4,9- | | | | |
| | triazaspiro[5.5]undecane | | | | |
| 2(60) | 1-(2-phenylethyl)-2,5-dioxo- | E | ESI (Pos., 20 V) | 2.94 | 342 (M + H)⁺ |
| | 3,4-propano-9-methyl-1,4,9- | | | | |
| | triazaspiro[5.5]undecane | | | | |

TABLE 1B-7

| Example | | HPLC | | Retention | |
|---|---|---|---|---|---|
| Number | Compund Name | condition | Mass condition | time (min) | Mass data |
| 2(61) | 1-propyl-2,5-dioxo-3,4- | B | APCI (Pos., 40 V) | 6.6 | 280 (M + H)⁺ |
| | propano-9-methyl-1,4,9- | | | | |
| | triazaspiro[5.5]undecane | | | | |
| 2(62) | 1-(1-benzyl-3-pyrrolidinyl)- | E | ESI (Pos., 20 V) | 2.83 | 397 (M + H)⁺ |
| | 2,5-dioxo-3,4-propano-9- | | | | |
| | methyl-1,4,9- | | | | |
| | triazaspiro[5.5]undecane | | | | |
| 2(63) | (3S)-1-propyl-2,5-dioxo-3,4- | D | APCI (Pos., 40 V) | 3.90 | 532 (M + H)⁺ |
| | ((2R)-2-benzyloxy-1,3- | | | | |
| | propano)-9-(6-phenylhexyl)- | | | | |
| | 1,4,9- | | | | |
| | triazaspiro[5.5]undecane | | | | |
| 2(64) | (3R)-1-propyl-2,5-dioxo-3,4- | D | APCI (Pos., 40 V) | 4.16 | 444 (M + H)⁺, |
| | (2-thiapropano)-9-(6- | | | | 398 |
| | phenylhexyl)-1,4,9- | | | | |
| | triazaspiro[5.5]undecane | | | | |

### REFERENCE EXAMPLE 7

### 1-benzyl-4-[N-butyl-N-(1-t-butoxycarbonylamino)cyclopentylcarbonyl]amino-4-[(2-morpholinoethyl)amino]carbonylpiperidine

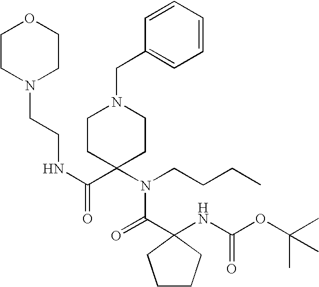

To a solution of 1-(t-butoxycarbonylamino)cyclopentanecarbonic acid (2.0 g), 1-benylpiperidone (1.6 ml) and n-butylamine (1.1 ml) in methanol (70 ml) was added 2-morpholinoethylisocyanide (1.2 ml). The mixture was stirred for overnight at 60° C. The mixture was concentrated, and diluted with ethyl acetate. The organic layer was washed with water, saturated aqueous solution of sodium hydrogen carbonate and brine, dried over anhydrous sodium sulfate, and concentrated. The obtained solid was washed with t-butyl methyl ether and filtrated to give the crude title compound (2.03 g). The obtained compound used next step without purification.

### EXAMPLE 3

### 1-butyl-2,5-dioxo-3,3-butano-9-benzyl-1,4,9-triazaspiro[5.5]undecane

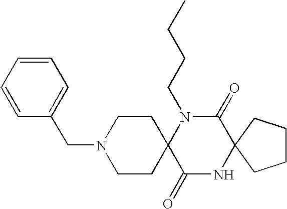

To a solution of the compound prepared in Reference Example 7 (1.56 g) in dichloromethane (5 ml) was added trifluoroacetic acid (5 ml) at 0° C. The mixture was stirred for 15 minute at same temperature, and stirred for overnight at room temperature. To the reaction mixture was added saturated aqueous solution of sodium hydrogen carbonate, extracted with dichloromethane. The extract was dried over anhydrous sodium sulfate, and concentrated. The obtained solid was washed with t-butyl methyl ether and filtrated to give the title compound (674 mg) having the following physical data. TLC: 0.56 (chloroform:methanol=10:1);

NMR(CD₃OD): δ 7.34-7.27 (m, 5H), 3.58 (s, 2H), 2.86-2.84 (m, 4H), 2.30-2.11 (m, 4H), 1.88-1.70 (m, 8H), 1.58-1.47 (m, 2H), 1.42-1.32 (m, 2H), 0.95 (t, J=7.5 Hz, 3H).

### EXAMPLE 4

### 1-butyl-2,5-dioxo-3,3-butano-1,4,9-triazaspiro[5.5]undecane hydrochloride

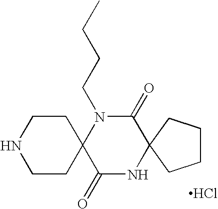

To a solution of the compound prepared in Example 3 (674 mg) in ethanol (18 ml) was added 20% palladium hydroxide on carbon (135 mg) under an atmosphere of argon. Under an atmosphere of hydrogen, the reaction mixture was stirred for overnight at 45° C. The reaction mixture was cooled to room temperature, replaced under an atmosphere of argon and filtrated through Celite (brand name). The filtrate was added 4N hydrochloric acid/ethyl acetate (1 ml), concentrated to give the title compound (569 mg) having the following physical data. TLC: 0.60 (t-butanol: acetic acid:water=4:2:1);

NMR(CD₃OD): δ 3.83-3.73 (m, 2H), 3.42-3.36 (m, 4H), 2.38-2.22 (m, 4H), 2.13-2.08 (m, H), 1.85-1.72 (m, 6H), 1.61-1.50 (m, 2H), 1.45-1.33 (m, 2H), 0.97 (t, J=7.5 Hz, 3H).

### EXAMPLE 5

### 1-butyl-2,5-dioxo-3,3-butano-9-[4-(4methylcarbamoylphenoxy)benzyl]-1,4,9-triazaspiro[5.5]undecane hydrochloride

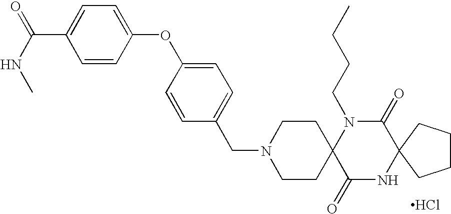

A solution of the compound prepared in Example 4 (100 mg) and 4-(4-methylcarbamoylphenoxy)benzaldehyde (85 mg) in 1% acetic acid/dimethylformamide solution (2 ml) was stirred for 2 hours. To the mixture was added sodium triacetoxyborohydride (96 mg). The mixture was stirred for overnight and concentrated. The residue was purified by silica gel column chromatography (ethyl acetate:methanol=30:1→15:1). To the obtained compound was added 4N hydrochloric acid/ethyl acetate (1 ml) and concentrated. The obtained solid was washed with t-butyl methyl ether, and filtrated to give the title compound (121 mg) having the following physical data. TLC: 0.51(ethyl acetate: methanol=4:1);

NMR (d₆-DMSO): δ 10.89 (br-s, 1H), 8.59 (s, 1H), 8.40 (br-d, J=4.5 Hz, 1H), 7.87 (d, J=8.7 Hz, 2H), 7.68 (d, J=8.7 Hz, 2H), 7.13 (d, J=8.7 Hz, 2H), 7.07 (d, J=8.7 Hz, 2H), 4.32 (m, 2H), 3.65-3.45 (m, 2H), 3.44-3.30 (m, 4H), 2.76 (d, J=4.5 Hz, 2H), 2.56-2.42 (m, 2H), 2.08-1.97 (m, 4H), 1.80-1.60 (m, 6H), 1.45-1.35 (m, 2H), 1.34-1.20 (m, 2H), 0.88 (t, J=7.5 Hz, 3H).

### FORMULATION EXAMPLE 1

The following components were admixed in a conventional technique, punched out to give 100 tablets each containing 50 mg of active ingredient.

| | |
|---|---|
| (3R)-1-Butyl-2,5-dioxo-3,4-(2-thiapropano)-9-[(1,4- | 5.0 g |
| benzodioxan-6-yl)methyl]-1,4,9-triazaspiro[5.5]undecane | |
| hydrochloride | |
| Calcium carboxymethylcellulose (disintegrant) | 0.2 g |
| Magnesium stearate (lubricant) | 0.1 g |
| Microcrystalline cellulose | 4.7 g |

### FORMULATION EXAMPLE 2

The following components were admixed in a conventional technique. The solution was sterilized in a conventional technique, filled in ampoules 5 ml each and freeze-dried in a conventional technique to give 100 ampoules each containing 20 mg of active ingredient.

| | | |
|---|---|---|
| (3R)-1-Butyl-2,5-dioxo-3,4-(2-thiapropano)-9-[(1,4- | 2.0 | g |
| benzodioxan-6-yl)methyl]-1,4,9-triazaspiro[5.5]undecane | | |
| hydrochloride | | |
| Mannitol | 20 | g |
| Distilled water | 500 | ml |

1. A spiroheterocyclic ring derivatives of the formula (I) 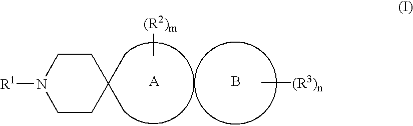
wherein R¹ is:
(1) hydrogen,
(2) C1-18 alkyl,
(3) C2-18 alkenyl,
(4) C2-18 alkynyl,
(5) —COR⁶,
(6) —CONR⁷R⁸,
(7) —COOR⁹,
(8) —SO₂R¹⁰,
(9) —COCOOR¹¹,
(10) —CONR¹²COR¹³,
(11) Cyc 1, or
(12) C1-18 alkyl, C2-18 alkenyl or C2-18 alkynyl substituted by 1-5 substituent(s) selected from (a) halogen, (b) —CONR⁷R⁸, (c) —COOR⁹, (d) —OR¹⁴, (e) —SR¹⁵, (f) —NR¹⁶R¹⁷, (g) —NR¹⁸COR¹⁹, (h) —SO₂NR²⁰R²¹, (i) —OCOR²², (j)—NR²³SO₂R²⁴, (k) —NR²⁵COOR²⁶, (l)—NR²⁷CONR²⁸R²⁹, (m) Cyc 1, (n) keto or (o) —N(SO₂R²⁴)₂,
wherein R⁶-R⁹, R¹¹-R²¹, R²³, R²⁵ and R²⁷-R²⁹ are each independently:
(1) hydrogen,
(2) C1-8 alkyl,
(3) C2-8 alkenyl,
(4) C2-8 alkynyl,
(5) Cyc 1, or
(6) C1-8 alkyl, C2-8 alkenyl or C2-8 alkynyl substituted by 1-5 substituent(s) selected from (a) Cyc 1, (b) halogen, (c) —OR³⁰, (d) —SR³¹, (e) —NR³²R³³, (f) —COOR³⁴, (g) —CONR³⁵R³⁶, (h) NR³⁷COR³⁸, (i) —NR³⁹SO₂R⁴ or (j)—N(SO₂R⁴⁰)₂, or
R⁷ and R⁸, R²⁰ and R²¹, R²⁸ and R²⁹, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁵—(C2-6 alkylene)-,
R¹⁹⁵ is hydrogen, C1-8 alkyl, phenyl or C1-8 alkyl substituted by phenyl,
R¹⁰, R²², R²⁴ and R²⁶ are each independently:
(1) C1-8 alkyl,
(2) C2-8 alkenyl,
(3) C2-8 alkynyl,
(4) Cyc 1, or
(5) C1-8 alkyl, C2-8 alkenyl or C2-8 alkynyl substituted by 1-5 substituent(s) selected from (a) Cyc 1, (b) halogen, (c) —OR³⁰, (d) —SR³¹, (e) —NR³²R³³, (f) —COOR³⁴, (g) —CONR³⁵R³⁶, (h) —NR³⁷COR³⁸, (i) —NR³⁹SO₂R⁴⁰ or (j) —N(SO₂R⁴⁰)₂,
R³⁰—R³⁷ and R³⁹ are each independently, hydrogen, C1-8 alkyl, Cyc 1 or C1-8 alkyl substituted by Cyc 1, or
R³⁵ and R³⁶, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁶—(C2-6 alkylene)-,
R¹⁹⁶ is hydrogen, C1-8 alkyl, phenyl or C1-8 alkyl substituted by phenyl,
R³⁸ and R⁴⁰ are each independently C1-8 alkyl, Cyc 1 or C1-8 alkyl substituted by Cyc 1,
Cyc 1 is a C3-15 mono, bi- or tri-(fused or spiro)carbocyclic ring or a 3-15 membered mono-, bi- or tri-(fused or spiro)cyclic hetero ring containing 1-4 nitrogen atom(s), 1-3 oxygen atom(s) and/or 1-3 sulfur atom(s),
Cyc 1 may be substituted by 1-5 of R⁵¹,
R⁵¹ is:
(1) C1-8 alkyl,
(2) C2-8 alkenyl,
(3) C2-8 alkynyl,
(4) halogen,
(5) nitro,
(6) trifluoromethyl,
(7) trifluoromethoxy,
(8) nitrile,
(9) keto,
(10) Cyc 2
(11)—OR⁵²,
(12) —SR⁵³,
(13) —NR⁵⁴R⁵⁵,
(14) —COOR⁵⁶,
(15)—CONR⁵⁷R⁵⁸,
(16) —NR⁵COR⁶⁰,
(17) —SO₂NR⁶¹R⁶²,
(18)—OCOR⁶³,
(19) NR⁶¹SO₂R⁶²,
(20) —NR⁶⁶COOR⁶⁷,
(21) —NR⁶⁸CONR⁶⁹R⁷⁰,
(22) —B(OR⁷¹)₂,
(23) —SO₂R⁷²,
(24) —N(SO₂R 72)₂, or
(25) C1-8 alkyl, C2-8 alkenyl or C2-8 alkynyl substituted by 1-5 substituent(s) selected from (a) halogen, (b) Cyc 2, (c) —OR⁵², (d) —SR⁵³, (e) —NR⁵⁴R⁵⁵, (f) —COOR⁵⁶, (g) —CONR⁵⁷R⁵⁸, (h) —NR⁵⁹COR⁶⁰, (i) —SO₂NR⁶¹R⁶², (j)—OCOR⁶³, (k) —NR⁶⁴SO₂R⁶⁵, (1)—NR⁶⁶COOR⁶⁷, (m) —NR⁶⁸CONR⁶⁹R⁷⁰, (n) —B(OR⁷¹)₂, (o) —SO₂R⁷², (p) —N(SO₂R⁷²)₂ or (q) keto,
R⁵²-R⁶², R⁶⁴, R⁶⁶ and R⁶⁸-R⁷¹ are each independently 1) hydrogen, 2) C1-8 alkyl, 3) C2-8 alkenyl, 4) C2-8 alkynyl, 5) Cyc 2 or 6) C1-8 alkyl, C2-8 alkenyl or C2-8 alkynyl substituted by Cyc 2, —OR⁷³, —COOR⁷⁴ or —NR⁷⁵R⁷⁶, or
R⁵⁷ and R⁵⁸, R⁶¹ and R⁶², R⁶⁹ and R⁷⁰, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁷—(C2-6 alkylene)-,
R¹⁹⁷ is hydrogen, C1-8 alkyl, phenyl or C1-8 alkyl substituted by phenyl,
R⁶³, R⁶⁵, R⁶⁷ and R⁷² are each independently 1) C1-8 alkyl, 2) C2-8 alkenyl, 3) C2-8 alkynyl, 4) Cyc 2 or 5) C1-8 alkyl, C2-8 alkenyl or C2-8 alkynyl substituted by Cyc 2, —OR⁷³, —COOR⁷⁴ or —NR⁷⁵R⁷⁶,
R⁷³—R⁷⁶ are independently hydrogen, C1-8 alkyl, Cyc 2 or C1-8 alkyl substituted by Cyc 2,
Cyc 2 has the same meaning as Cyc 1,
Cyc 2 may be substituted by 1-5 of R⁷⁷,
R⁷⁷ is:
1) C1-8 alkyl,
2) halogen,
3) nitro,
4) trifluoromethyl,
5) trifluoromethoxy,
6) nitrile,
7) —OR⁷⁸,
8) —NR⁷⁹R⁸⁰,
9) —COOR⁸¹,
10) —SR⁸²,
11) —CONR⁸³R⁸⁴,
12) C2-8 alkenyl,
13) C2-8 alkynyl,
14) keto,
15) Cyc 6,
16) NR¹⁶¹COR¹⁶²,
17) —SO₂NR¹⁶³R¹⁶⁴,
18) —OCOR¹⁶⁵,
19) —NR¹⁶⁶SO₂R¹⁶⁷,
20) —NR¹⁶⁸COOR¹⁶⁹,
21) —NR¹⁷⁰CONR¹⁷¹R¹⁷²,
22) —SO₂R¹⁷³,
23) —N(SO₂R¹⁶⁷)₂, or
24) C1-8 alkyl, C2-8 alkenyl or C2-8 alkynyl substituted by 1-5 substituent(s) selected from (a) halogen, (b) —OR⁷⁸, (c) —NR⁷⁹R⁸⁰, (d) —COOR⁸¹, (e) —SR⁸², (f) —CONR⁸³R⁸⁴, (g) keto, (h) Cyc 6, (i) —NR¹⁶¹COR¹⁶², (j) —SO₂NR¹⁶³R¹⁶⁴, (k) —OCOR¹⁶⁵, (l) —NR¹⁶⁶SO₂R¹⁶⁷, (m) —NR¹⁶⁸COOR¹⁶⁹, (n) —NR¹⁷⁰CONR¹⁷¹R¹⁷², (o) —SO₂R or (p) —N(SO₂R¹⁶⁷)₂,
R⁷⁸-R⁸⁴, R¹⁶¹-R⁶⁴, R⁶⁶, R¹⁶⁸ and R¹⁷⁰-R¹⁷² are each independently (a) hydrogen, (b) C1-8 alkyl, (c) C2-8 alkenyl, (d) C2-8 alkynyl, (e) Cyc 6 or (f) C1-8 alkyl, C2-8 alkenyl or C2-8 alkynyl substituted by Cyc 6, —OR¹⁷⁴, —COOR¹⁷⁵, —NR¹⁷⁶R¹⁷⁷ or —CONR¹⁷⁸R¹⁷⁹, or
R⁸³ and R⁸⁴, R¹⁶³ and R⁶⁴, R¹⁷¹ and R⁷², taken together, are 1) C2-6 alkylene, 2)—(C2-6 alkylene)-O—(C2-6 alkylene)-, 3) —(C2-6 alkylene)-S—(C2-6 alkylene)- or 4) —(C2-6 alkylene)-NR¹⁹⁸—(C2-6 alkylene)-,
R¹⁹⁸ is hydrogen, C1-8 alkyl, phenyl or C1-8 alkyl substituted by phenyl,
R¹⁶⁵, R¹⁶⁷, R¹⁶⁹ and R¹⁷³ are each independently (a) C1-8 alkyl, (b) C2-8 alkenyl, (c) C2-8 alkynyl, (d) Cyc 6 or (e) C1-8 alkyl, C2-8 alkenyl or C2-8 alkynyl substituted by Cyc 6, —OR¹⁷⁴, —COOR¹⁷⁵, —NR¹⁷⁶R¹⁷⁷ or —CONR¹⁷⁸R¹⁷⁹,
R¹⁷⁴-R¹⁷⁷ are each independently (1) hydrogen, (2) C1-8 alkyl, (3) Cyc 6 or (4) C1-8 alkyl substituted by Cyc 6, or
R¹⁷⁸ and R¹⁷⁹, taken together, are 1) C2-6 alkylene, 2) —(C2-6 alkylene)-O—(C2-6 alkylene)-, 3) —(C2-6 alkylene)-S—(C2-6 alkylene)- or 4) —(C2-6 alkylene)-NR¹⁹⁹—(C2-6 alkylene)-,
R¹⁹⁹ is hydrogen, C1-8 alkyl, phenyl or C1-8 alkyl substituted by phenyl,
Cyc 6 is a C3-8 mono-carbocyclic ring or a 3-8 membered mono-cyclic hetero ring containing 1-4 nitrogen atom(s), 1-2 oxygen atom(s) and/or 1-2 sulfur atom(s),
Cyc 6 may be substituted by 1-5 of R¹⁸⁰,
R¹⁸⁰ is:
(1) C1-8 alkyl,
(2) halogen,
(3) nitro,
(4) trifluoromethyl,
(5) trifluoromethoxy,
(6) nitrile,
(7) —OR¹⁸¹,
(8) —NR¹⁸²R¹⁸³,
(9) —COOR¹⁸⁴,
(10) —SR¹⁸⁵, or
(11) —CONR¹⁸⁶R¹⁸⁷,
R¹⁸¹-R¹⁸⁷ are each independently (1) hydrogen, (2) C1-8 alkyl, (3) phenyl or (4) C1-8 alkyl substituted by phenyl, or
R¹⁸² and R¹⁸³, R¹⁸⁶ and R⁸⁷, taken together, are (1) C2-6 alkylene, (2) —(C2-6 alkylene)-O—(C2-6 alkylene)-, (3) —(C2-6 alkylene)-S—(C2-6 alkylene)- or (4) —(C2-6 alkylene)-NR²⁰⁰—(C2-6 alkylene)-,
R²⁰⁰ is hydrogen, C1-8 alkyl, phenyl or C1-8 alkyl substituted by phenyl, 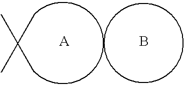
is (i) a fused bi-cyclic ring which A ring and B ring bound by two atoms or (ii) a spiro ring which A ring and B ring bound by spiro,
A ring is (i) a C5 or 6 partially or fully saturated carbocyclic ring or (ii) a 5 or 6 membered partially or fully saturated hetero ring containing 1-3 hetero atom(s) selected from a nitrogen atom(s), an oxygen atom(s) and/or a sulfur atom(s),
B ring is (i) a C4-7 partially or fully saturated carbocyclic ring or (ii) a 4-7 membered partially or fully saturated hetero ring containing 1-3 hetero atom(s) selected from a nitrogen atom(s), an oxygen atom(s) and/or a sulfur atom(s),
R² is:
(1) keto,
(2) thioketo,
(3) C1-8 alkyl,
(4) C2-8 alkenyl,
(5) C2-8 alkynyl,
(6) —OR⁹⁰,
(7) Cyc 3, or
(8) C1-8 alkyl, C2-8 alkenyl or C2-8 alkynyl substituted by 1-5 substituent(s) selected from (a) halogen, (b) —OR⁹⁰, (c) —SR⁹¹, (d) —NR⁹²R⁹³, (e) —COOR⁹⁴, (f) —CONR⁹⁵R⁹⁶, (g) —NR⁹⁷COR⁹⁸, (h) —SO₂NR⁹⁹R¹⁰⁰, (i) —OCOR¹⁰¹, (j)—NR¹⁰²SO₂R¹⁰³, (k) —NR¹⁰⁴COOR¹⁰⁵, (l) —NR¹⁰⁶CONR¹⁰⁷R¹⁰⁸, (m) Cyc 3, (n) keto or (o) —N(SO₂R¹⁰³)₂,
R⁹⁰-R¹⁰⁰, R¹⁰², R¹⁰⁴ and R¹⁰⁶-R¹⁰⁸ are each independently (1) hydrogen, (2) C1-8 alkyl, (3) C2-8 alkenyl, (4) C2-8 alkynyl, (5) Cyc 3 or (6) C1-8 alkyl, C2-8 alkenyl or C2-8 alkynyl substituted by Cyc 3, or
R⁹⁵ and R⁹⁶, R⁹⁹ and R¹⁰⁰, R¹⁰⁷ and R¹⁰⁸, taken together, are (1) C2-6 alkylene, (2)—(C2-6 alkylene)-O—(C2-6 alkylene)-, (3) —(C2-6 alkylene)-S—(C2-6 alkylene)- or (4) —(C2-6 alkylene)-NR22—(C2-6 alkylene)-,
R²⁰² is hydrogen, C1-8 alkyl, phenyl or C1-8 alkyl substituted by phenyl, R¹⁰¹, R¹⁰³ and R¹⁰⁵ are each independently (1) C1-8 alkyl, (2) C2-8 alkenyl, (3) C2-8 alkynyl or (4) Cyc 3, or (5) C1-8 alkyl, C2-8 alkenyl or C2-8 alkynyl substituted by Cyc 3,
Cyc 3 has the same meaning as Cyc 1,
Cyc 3 may be substituted by 1-5 of R¹⁰⁹,
R¹⁰⁹ has the same meaning as R⁵¹,
R³ is:
(1) C1-8 alkyl,
(2) C2-8 alkenyl,
(3) C2-8 alkynyl,
(4) —COOR¹²⁰,
(5) —CONR¹²¹R¹²²,
(6) Cyc 4, or
(7) —OR¹²³,
(8)—COR¹³¹,
(9) —SO₂R¹³³, or
(10) C1-8 alkyl, C2-8 alkenyl or C2-8 alkynyl substituted by a substituent(s) selected from (a) halogen, (b) nitrile, (c) Cyc 4, (d) —COOR¹²⁰, (e) —CONR¹²¹R¹²², (f) —OR¹²³, (g) —SR¹²⁴, (h) —NR¹²⁵R¹²⁶, (i) —NR¹²⁷COR¹²⁸, (j) —SO₂NR¹²⁹R¹³⁰, (k) —OCOR¹³¹, (l) —NR¹³²SO₂R¹³³, (m) —NR¹³⁴COOR¹³⁵, (n) —NR¹³⁶CONR¹³⁷R¹³⁸ or (o) keto,
R¹²⁰-R¹³⁰, R¹³², R¹³⁴ and R¹³⁶-R¹³⁸ are each independently (1) hydrogen, (2) C1-8 alkyl, (3) C2-8 alkenyl, (4) C2-8 alkynyl, (5) Cyc 4 or (6) C1-8 alkyl, C2-8 alkenyl or C2-8 alkynyl substituted by Cyc 4, halogen, —OR¹⁴⁸, —SR¹⁴⁹, —COOR¹⁵⁰ or —NHCOR¹⁴¹, or
R¹²¹ and R¹²², R¹²⁹, and R¹³⁰, R¹³⁷ and R¹³⁸, taken together, are 1) C2 alkylene, 2) (C2-6 alkylene)-O—(C2-6 alkylene)-, 3) —(C2-6 alkylene)-S—(C2-6 alkylene)- or 4) —(C2-6 alkylene)-NR²⁰²—(C2-6 alkylene)-,
R²⁰² is hydrogen, C1-8 alkyl, phenyl or C1-8 alkyl substituted by phenyl,
R¹³¹, R¹³³ and R¹³⁵ are each independently (1) C1-8 alkyl, (2) C2-8 alkenyl, (3) C2-8 alkynyl, (4) Cyc 4 or (5) C1-8 alkyl, C2-8 alkenyl or C2-8 alkynyl substituted by Cyc 4, halogen, —OR¹⁴⁸, —SR¹⁴⁹, —COOR¹⁵⁰ or —NHCOR¹⁴¹,
R¹⁴¹ is (1) C1-8 alkyl, (2) C2-8 alkenyl, (3) C2-8 alkynyl, (4) Cyc 4 or (5) C1-8 alkyl, C2-8 alkenyl or C2-8 alkynyl substituted by Cyc 4,
R¹⁴⁸-R¹⁵⁰ are each independently (1) hydrogen, (2) C1-8 alkyl, (3) C2-8 alkenyl, (4) C2-8 alkynyl, (5) Cyc 4 or (6) C1-8 alkyl, C2-8 alkenyl or C2-8 alkynyl substituted by Cyc 4,
Cyc 4 has the same meaning as Cyc 1,
Cyc 4 may be substituted by 1-5 of R¹⁴⁴,
R¹⁴⁴ has the same meaning as R⁵¹,
m is 0-5,
n is 0-5,
when m is 2-5, then R² of m are the same or different,
when n is 2-5, then R³ of n are the same or different,
a quaternary ammonium salt thereof, an N-oxides thereof or a non-toxic salt thereof.

2. The compound according to claim 1, which is
(1) (3R)-1-butyl-2,5-dioxo-3,4-(2-thiapropano)-9-[(1,4-benzodioxan-6-yl)methyl]-1,4,9-triazaspiro[5.5]undecane,
(2) (3R)-1-butyl-2,5-dioxo-3,4-(2-thiapropano)-9-[(4-phenoxyphenyl)methyl]-1,4,9-triazaspiro[5.5]undecane,
(3) (3S)-1-(2-methylpropyl)-2,5-dioxo-3,4-propano-9-(2-phenylethyl)-1,4,9-triazaspiro[5.5]undecane,
(4) (3S)-1-(1-benzyl-4-pyperidinyl)-2,5-dioxo-3,4-propano-9-(2-phenylethyl)-1,4,9-triazaspiro[5.5]undecane,
(5) (3S)-1-(2,2-diphenylpropyl)-2,5-dioxo-3,4-propano-9-(2-phenylethyl)-1,4,9-triazaspiro[5.5]undecane,
(6) 1-(2-furanylmethyl)-2,5-dioxo-3,4-propano-9-benzyl-1,4,9-triazaspiro[5.5]undecane,
(7) 1-(2-tetrahydrofuranylmethyl)-2,5-dioxo-3,4-propano-9-benzyl-1,4,9-triazaspiro[5.5]undecane,
(8) 1-(2-(3-indole)ethyl)-2,5-dioxo-3,4-propano-9-benzyl-1,4,9-triazaspiro[5.5]undecane,
(9) 1,9-dibenzyl-2,5-dioxo-3,4-propano-1,4,9-triazaspiro[5.5]undecane,
(10) 1-(2,2-diphenylethyl)-2,5-dioxo-3,4-propano-9-benzyl-1,4,9-triazaspiro[5.5]undecane,
(11) 1-(2-phenylethyl)-2,5-dioxo-3,4-propano-9-benzyl-1,4,9-triazaspiro[5.5]undecane,
(12) 1-propyl-2,5-dioxo-3,4-propano-9-benzyl-1,4,9-triazaspiro[5.5]undecane,
(13) 1-(1-benzyl-3-pyrrolidinyl)-2,5-dioxo-3,4-propano-9-benzyl-1,4,9-triazaspiro[5.5]undecane,
(14) 1-(2-tetrahydrofuranylmethyl)-2,5-dioxo-3,4-propano-9-(2-phenylethyl)-1,4,9-triazaspiro[5.5]undecane,
(15) 1-(2-(3-indole)ethyl)-2,5-dioxo-3,4-propano-9-(2-phenylethyl)-1,4,9-triazaspiro[5.5]undecane,
(16) 1-benzyl-2,5-dioxo-3,4-propano-9-(2-phenylethyl)-1,4,9-triazaspiro[5.5]undecane,
(17) 1-(2,2-diphenylethyl)-2,5-dioxo-3,4-propano-9-(2-phenylethyl)-1,4,9-triazaspiro[5.5]undecane,
(18) 1-(2-phenylethyl)-2,5-dioxo-3,4-propano-9-(2-phenylethyl)-1,4,9-triazaspiro[5.5]undecane,
(19) 1-propyl-2,5-dioxo-3,4-propano-9-(2-phenylethyl)-1,4,9-triazaspiro[5.5]undecane,
(20) 1-(1-benzyl-3-pyrrolidinyl)-2,5-dioxo-3,4-propano-9-(2-phenylethyl)-1,4,9-triazaspiro[5.5]undecane,
(21) 1-(2-furanylmethyl)-2,5-dioxo-3,4-propano-9-(3-phenylpropyl)-1,4,9-triazaspiro[5.5]undecane,
(22) 1-(2-tetrahydrofuranylmethyl)-2,5-dioxo-3,4-propano-9-(3-phenylpropyl)-1,4,9-triazaspiro[5.5]undecane,
(23) 1-(2-(3-indole)ethyl)-2,5-dioxo-3,4-propano-9-(3-phenylpropyl)-1,4,9-triazaspiro[5.5]undecane,
(24) 1-benzyl-2,5-dioxo-3,4-propano-9-(3-phenylpropyl)-1,4,9-triazaspiro[5.5]undecane,
(25) 1-(2,2-diphenylethyl)-2,5-dioxo-3,4-propano-9-(3-phenylpropyl)-1,4,9-triazaspiro[5.5]undecane,
(26) 1-(2-phenylethyl)-2,5-dioxo-3,4-propano-9-(3-phenylpropyl)-1,4,9-triazaspiro[5.5]undecane,
(27) 1-propyl-2,5-dioxo-3,4-propano-9-(3-phenylpropyl)-1,4,9-triazaspiro[5.5]undecane,
(28) 1-(1-benzyl-3-pyrrolidinyl)-2,5-dioxo-3,4-propano-9-(3-phenylpropyl)-1,4,9-triazaspiro[5.5]undecane,
(29) 1-(2-furanylmethyl)-2,5-dioxo-3,4-propano-9-(4-phenylbutyl)-1,4,9-triazaspiro[5.5]undecane,
(30) 1-(2-tetrahydrofuranylmethyl)-2,5-dioxo-3,4-propano-9-(4-phenylbutyl)-1,4,9-triazaspiro[5.5]undecane,
(31) 1-(2-(3-indole)ethyl)-2,5-dioxo-3,4-propano-9-(4-phenylbutyl)-1,4,9-triazaspiro[5.5]undecane,
(32) 1-benzyl-2,5-dioxo-3,4-propano-9-(4-phenylbutyl)-1,4,9-triazaspiro[5.5]undecane,
(33) 1-(2,2-diphenylethyl)-2,5-dioxo-3,4-propano-9-(4-phenylbutyl)-1,4,9-triazaspiro[5.5]undecane,
(34) 1-(2-phenylethyl)-2,5-dioxo-3,4-propano-9-(4-phenylbutyl)-1,4,9-triazaspiro[5.5]undecane,
(35) 1-propyl-2,5-dioxo-3,4-propano-9-(4-phenylbutyl)-1,4,9-triazaspiro[5.5]undecane,
(36) 1-(1-benzyl-3-pyrrolidinyl)-2,5-dioxo-3,4-propano-9-(4-phenylbutyl)-1,4,9-triazaspiro[5.5]undecane,
(37) 1-(2-furanylmethyl)-2,5-dioxo-3,4-propano-9-phenyl-1,4,9-triazaspiro[5.5]undecane,
(38) 1-(2-tetrahydrofuranylmethyl)-2,5-dioxo-3,4-propano-9-phenyl-1,4,9-triazaspiro[5.5]undecane,
(39) 1-(2-(3-indole)ethyl)-2,5-dioxo-3,4-propano-9-phenyl-1,4,9-triazaspiro[5.5]undecane,
(40) 1-benzyl-2,5-dioxo-3,4-propano-9-phenyl-1,4,9-triazaspiro[5.5]undecane,
(41) 1-(2,2-diphenylethyl)-2,5-dioxo-3,4-propano-9-phenyl-1,4,9-triazaspiro[5.5]undecane,
(42) 1-(2-phenylethyl)-2,5-dioxo-3,4-propano-9-phenyl-1,4,9-triazaspiro[5.5]undecane,
(43) 1-propyl-2,5-dioxo-3,4-propano-9-phenyl-1,4,9-triazaspiro[5.5]undecane,
(44) 1-(2-tetrahydrofuranylmethyl)-2,5-dioxo-3,4-propano-9-(5-phenylpentyl)-1,4,9-triazaspiro[5.5]undecane,
(45) 1-(2-(3-indole)ethyl)-2,5-dioxo-3,4-propano-9-(5-phenylpentyl)-1,4,9-triazaspiro[5.5]undecane,
(46) 1-benzyl-2,5-dioxo-3,4-propano-9-(5-phenylpentyl)-1,4,9-triazaspiro[5.5]undecane,
(47) 1-(2,2-diphenylethyl)-2,5-dioxo-3,4-propano-9-(5-phenylpentyl)-1,4,9-triazaspiro[5.5]undecane,
(48) 1-(2-phenylethyl)-2,5-dioxo-3,4-propano-9-(5-phenylpentyl)-1,4,9-triazaspiro[5.5]undecane,
(49) 1-propyl-2,5-dioxo-3,4-propano-9-(5-phenylpentyl)-1,4,9-triazaspiro[5.5]undecane,
(50) 1-(1-benzyl-3-pyrrolidinyl)-2,5-dioxo-3,4-propano-9-(5-phenylpentyl)-1,4,9-triazaspiro[5.5]undecane,
(51) 1-(2-tetrahydrofuranylmethyl)-2,5-dioxo-3,4-propano-9-(6-phenylhexyl)-1,4,9-triazaspiro[5.5]undecane,
(52) 1-(2-(3-indole)ethyl)-2,5-dioxo-3,4-propano-9-(6-phenylhexyl)-1,4,9-triazaspiro[5.5]undecane,
(53) 1-benzyl-2,5-dioxo-3,4-propano-9-(6-phenylhexyl)-1,4,9-triazaspiro[5.5]undecane,
(54) 1-(2,2-diphenylethyl)-2,5-dioxo-3,4-propano-9-(6-phenylhexyl)-1,4,9-triazaspiro[5.5]undecane,
(55) 1-(2-phenylethyl)-2,5-dioxo-3,4-propano-9-(6-phenylhexyl)-1,4,9-triazaspiro[5.5]undecane,
(56) 1-propyl-2,5-dioxo-3,4-propano-9-(6-phenylhexyl)-1,4,9-triazaspiro[5.5]undecane,
(57) 1-(1-benzyl-3-pyrrolidinyl)-2,5-dioxo-3,4-propano-9-(6-phenylhexyl)-1,4,9-triazaspiro[5.5]undecane,
(58) 1-(2-furanylmethyl)-2,5-dioxo-3,4-propano-9-methyl-1,4,9-triazaspiro[5.5]undecane,
(59) 1-(2-tetrahydrofuranylmethyl)-2,5-dioxo-3,4-propano-9-methyl-1,4,9-triazaspiro[5.5]undecane,
(60) 1-(2-(3-indole)ethyl)-2,5-dioxo-3,4-propano-9-methyl-1,4,9-triazaspiro[5.5]undecane,
(61) 1-benzyl-2,5-dioxo-3,4-propano-9-methyl-1,4,9-triazaspiro[5.5]undecane,
(62) 1-(2,2-diphenylethyl)-2,5-dioxo-3,4-propano-9-methyl-1,4,9-triazaspiro[5.5]undecane,
(63) 1-(2-phenylethyl)-2,5-dioxo-3,4-propano-9-methyl-1,4,9-triazaspiro[5.5]undecane,
(64) 1-propyl-2,5-dioxo-3,4-propano-9-methyl-1,4,9-triazaspiro[5.5]undecane,
(65) 1-(1-benzyl-3-pyrrolidinyl)-2,5-dioxo-3,4-propano-9-methyl-1,4,9-triazaspiro[5.5]undecane,
(66) (3S)-1-propyl-2,5-dioxo-3,4-((2R)-2-benzyloxy-1,3-propano)-9-(6-phenylhexyl)-1,4,9-triazaspiro[5.5]undecane,
(67) (3R)-1-propyl-2,5-dioxo-3,4-(2-thiapropano)-9-(6-phenylhexyl)-1,4,9-triazaspiro[5.5]undecane,
(68) 1-butyl-2,5-dioxo-3,3-butano-9-benzyl-1,4,9-triazaspiro[5.5]undecane,
(69) 1-butyl-2,5-dioxo-3,3-butano-1,4,9-triazaspiro[5.5]undecane, or
(70) 1-butyl-2,5-dioxo-3,3-butano-9-[4-(4-methylcarbamoylphenoxy)benzyl]-1,4,9-triazaspiro[5.5]undecane,
a quaternary ammonium salt thereof, an N-oxide thereof or a non-toxic salt thereof.

3. A pharmaceutical composition comprising the triazaspiro[5.5]undecane derivative of the formula (I) according to claim 1, a quaternary ammonium salt thereof, an N-oxide thereof or a non-toxic salt thereof, as an active ingredient, and a pharmaceutically acceptable carrier.

4. The pharmaceutical composition according to claim 3, which is a chemokine/chemokine regulator.

5. A method for prevention and/or treatment for asthma, atopic dermatitis, urticaria, allergic bronchopulmonary aspergillosis, allergic eosinophilic gastroenteritis, nephritis, nephropathy, hepatitis, arthritis, rheumatoid arthritis, psoriasis, rhinitis, conjunctivitis, ischemic reperfusion disorder, multiple sclerosis, ulcerative colitis, adult respiratory distress syndrome, cytotoxic shock, diabetes, autoimmune disease, multiple organ failure, immunosuppression, cancer metastasis and acquired immune deficiency syndrome, comprising administering to a subject in need thereof an effective amount of the triazaspiro[5.5]undecane derivative of the formula (I) according to claim 1, a quaternary ammonium salt thereof, an N-oxide thereof or a non-toxic salt thereof.
